SEQUENCE LISTING (7 sequences)

SEQ ID NO: 1 - protein, 283 aa, Homo sapiens
MARRPRAPAASGEEFSFVSPLVKYLLFFFNMLFWVISMVMVAVGVYARLMKHAEAALACLAVDPAILLIVVGVLMFLLTFCGCIGSLRENICLLQTFSLCLTAVFLLQLAAGILGFVFSDKARGKVSEIINNAIVHYRDDLDLQNLIDFGQKKFSCCGGISYKDWSQNMYFNCSEDNPSRERCSVPYSCCLPTPDQAVINTMCGQGMQAFDYLEASKVIYTNGCIDKLVNWIHSNLFLLGGVALGLAIPQLVGILLSQILVNQIKDQIKLQLYNQQHRADPWY

SEQ ID NO: 2 - protein, 283 aa, Mus musculus
MARRPGVPAAYGDEFSFVSPLVKYLLFFFNMLFWVISMVMVAVGVYARLMKHAEAALACLAVDPAILLIVVGVLMFLLTFCGCIGSLRENICLLQTFSLCLTIVFLLQLAAGILGFVFSDKARGKVSEIINNAIVHYRDDLDLQNLIDFGQKKFSCCGGISYRDWSQNMYFNCSEDNPSRERCSVPYSCCLPTPNQAVINTMCGQGMQALDYLEASKVIYTNGCIDKLVNWIHSNLFLLGGVALGLAIPQLVGILLSQVLVNQIKDQIKLQLYNQQHRADPWY

SEQ ID NO: 3 - protein, 274 aa, Artificial Sequence
  Consensus sequence
MARRPPAAGEFSFVSPLVKYLLFFFNMLFWVISMVMVAVGVYARLMKHAEAALACLAVDPAILLIVVGVLMFLLTFCGCIGSLRENICLLQTFSLCLTVFLLQLAAGILGFVFSDKARGKVSEIINNAIVHYRDDLDLQNLIDFGQKKFSCCGGISYDWSQNMYFNCSEDNPSRERCSVPYSCCLPTPQAVINTMCGQGMQADYLEASKVIYTNGCIDKLVNWIHSNLFLLGGVALGLAIPQLVGILLSQLVNQIKDQIKLQLYNQQHRADPWY

SEQ ID NO: 4 - DNA, 20 bp, Artificial Sequence
  tspan33 forward primer
caacatgctcttctgggtga

SEQ ID NO: 5 - DNA, 20 bp, Artificial Sequence
  tspan33 reverse primer
attagccgagcgtagacacc

SEQ ID NO: 6 - DNA, 27 bp, Artificial Sequence
  CD20 forward primer
aacaaaatctctactttgatggaactt

SEQ ID NO: 7 - DNA, 20 bp, Artificial Sequence
  CD20 reverse primer
gcaaggcctactgctgagtt

(19) United States
(12) Patent Application Publication
(10) Pub. No.: US 2016/0237152 A1
(43) Pub. Date: Aug. 18, 2016

(54) TSPAN 33 IS A CANDIDATE FOR ANTIBODY TARGETED THERAPY FOR THE TREATMENT OF B CELL HODGKIN LYMPHOMAS

(71) Applicants: THE REGENTS OF THE UNIVERSITY OF CALIFORNIA, Oakland, CA (US); Universidad Autonoma De Nuevo León, Nuevo León (MX)

(72) Inventors: Albert ZLOTNIK, San Diego, CA (US); Peter HEVEZI, Encinitas, CA (US); Van LUU, Tustin, CA (US); Juan Pablo FLORES, Nuevo Leon (MX)

(73) Assignees: The Regents of the University of California, Oakland, CA (US); Universidad Autonoma De Nuevo León, Nuevo León (MX)

(21) Appl. No.: 14/653,572

(22) PCT Filed: Dec. 20, 2013
(86) PCT No.: PCT/US13/77273

§ 371 Date: Jun. 18, 2015

Related U.S. Application Data
(60) Provisional application No. 61/740,946, filed on Dec. 21, 2012.

(51) Int. Cl. C07K 16/28; G01N 33/569; C12Q 1/68; C07K 16/30

(52) U.S. Cl. CPC C07K 16/28; C07K 16/3061; G01N 33/56972; C12Q 1/6883; C07K 2317/76; G01N 2333/705; C12Q 2600/158

(57) ABSTRACT

A method of treating a disease associated with activated B lymphocytes expressing Tetraspanin 33 (TSPAN33/BAAM). The disease can be, for example, lymphoma or an immune disease. The method includes administering an anti-TSPAN33/BAAM antibody to a patient in need of such treatment in an amount effective to treat the disease. Methods of purifying activated B cells and identifying activated and/or diseased B cells are also provided.

### STATEMENT REGARDING FEDERALLY SPONSORED RESEARCH OR DEVELOPMENT

This invention was made with Government support under Grant No. R21 AI096278 from the National Institutes of Health. The Government has certain rights in this invention.

### BACKGROUND

1. Field of the Invention

The present invention relates to the protein TSPAN33 which is expressed in activated B cells.

2. Related Art

B cells are lymphocytes that orchestrate the humoral response of the adaptive immune system (1). Unlike T cells that mature in the thymus, B cells develop in the bone marrow, where they mature into mature naïve B cells (1). B cells are solely responsible for secreting antibodies that recognize foreign antigens or, in the case of autoimmune diseases, autoantigens. Antibodies come in a variety of subtypes that determine both their location and function, such as IgA that participates in protection of mucosal surfaces. Certain types of lymphomas are of B cell origin. B cell lymphomas have historically been divided into two major types; Hodgkin lymphoma (HL) and non-Hodgkin lymphoma (NHL). Hodgkin's lymphoma, named after Thomas Hodgkin and first described in 1832 (2), is characterized by the presence of Reed Sternberg cells, enlargement of spleen, lymph node, or other immune tissue of the body, as well as abnormal growth that may spread beyond the lymphatic tissue. The term ‘Non-Hodgkin's lymphomas’ has been used to describe all types of lymphoma not presenting with the hallmark HL symptoms. Current lymphoma classification has superseded the HL or NHL grouping system with one containing 80 types in 4 broad categories (2). Some embodiments of the present invention involve using a novel biomarker expressed in the membrane of activated B cells or B cell lymphomas to identify specific diseased B cells or to achieve the specific elimination of diseased B cells or T cell lymphomas that express Tetraspanin 33 (TSPAN33), also known as the BAAM antigen, as some T cell lymphomas are known to aberrantly express B cell antigens, such as CD20 (3). Thus, use of BAAM as a therapeutic target is not restricted by lymphoma type but by the presence of the protein encoded by the TSPAN33/BAAM gene on the surface of lymphocytic cells.

Cancer immunotherapy has been transformed due to the development of therapeutic monoclonal antibodies. These antibodies target cell surface molecules specifically expressed in tumor cells. There are technologies, such as gene arrays, that allow the collective screening of expression of thousands of genes at a time. Application of bioinformatics allows the analysis of gene array data in order to identify genes encoding cell surface proteins that represent targets for the development of monoclonal antibodies. These antibodies can then be used as therapeutics to either slow the growth of tumors, or to directly kill tumor cells. Antibody targeted therapy has enjoyed increasing popularity, since Paul Ehrlich first envisioned antibodies as “magic bullets” that could deliver toxins to microbes or tumors in 1908 (4). In 1981 Gaffar, S. A., et al. (5) used radiolabeled antibodies against human carcinoembryonic antigen (CEA) to deliver specific cytoxicity, possibly through induction of DNA damage, to human colonic cancer xenografts. In 1988 DeNardo, et al. (6), reported complete or partial remission of 4 out of 10 patients with B cell malignancies, following the administration of radiolabeled antibody targeted therapy. Soon after, others have reported similar antitumor activity of “naked” (non-labeled) antibodies via complement mediated cytoxicity (CMC) or antibody dependent cellular cytotoxicity (ADCC) (7).

The binding of therapeutic antibody to the target molecule can trigger the signal transduction pathway normally controlled by the target molecule. This can lead to modifications of the fate of the tumor cell. It can cause apoptosis, necrosis, cell cycle arrest, enhanced proliferation, or differentiation. Some of these altered cell behaviors are desirable in the case of a cancer cell, especially those (necrosis, apoptosis) that lead to cell death or arrest of proliferation. People skilled in the art can determine whether a given antibody induces any of these effects in a tumor cell (8-9).

Monoclonal antibodies produced from mouse cells require ‘humanization’ to reduce their immunogenicity in order to be used in humans. There are several ways of doing this. One is by producing humanized antibodies where the mouse regions of the antibody (crystallizable fragment or Fc) are replaced with human Fc sequences (9). This can be done using a variety of molecular biology techniques (8-9). Alternatively, the antibodies can be produced by immunizing transgenic mice that have had their immune system altered by replacing mouse with human immunoglobulin genes using molecular biology techniques. Several such mice have been produced (7).

Given the possibilities described above, therapeutic monoclonal antibodies have become preferred methods to treat various cancers (10). FDA-approved antibody based therapies, such as rituximab (an anti-CD20 antibody), have been used for the treatment of non-Hodgkin's lymphoma (NHL) as well as autoimmune disorders, such as rheumatoid arthritis (RA) (11). Thus, antibody targeted therapy towards unique biomarkers expressed on disease cells/tissue has proven effective in treating human cancers or autoimmune disorders. Other examples include Herceptin, a humanized monoclonal antibody that targets the Her-2 antigen in breast cancer cells (12) or Avastin, a humanized antibody which targets vascular endothelial growth factor in colorectal cancers (13). These examples represent highly successful antibodies that have dramatic (positive) therapeutic effects in certain human cancers.

Antibodies that target B cells have proven therapeutically important because a number of lymphomas and leukemias express B cell antigens (11). An example is Rituximab (14), a therapeutic antibody that targets CD20, a protein expressed in certain human lymphomas. However, CD20, is also expressed by normal B cells, so although antibody therapy targeting CD20 eliminates most of the tumor cells, the treatment also ablates their normal B cells which also express CD20 (15). This is a serious side effect of the administration of rituximab in humans. Nevertheless, the benefit of eliminating tumor cells justifies the use of rituximab in patients with CD20 positive lymphomas (11).

### SUMMARY

In one aspect, a method of treating a lymphoma or leukemia in which TSPAN33 is upregulated is provided. The method includes administering an anti-TSPAN33 antibody to a patient in need of such treatment in an amount effective to treat the lymphoma or leukemia.

In the method:

a) the lymphoma can be a Hodgkin lymphoma, a non-Hodgkin lymphoma, precursor T-cell leukemia/lymphoma, follicular lymphoma, diffuse large B cell lymphoma, mantle cell lymphoma, B-cell chronic lymphocytic leukemia/lymphoma, MALT lymphoma, Burkitt's lymphoma, Burkitt's lymphoma, peripheral T-cell lymphoma-Not-Otherwise-Specified, nodular sclerosis form of Hodgkin lymphoma, or mixed-cellularity subtype of Hodgkin lymphoma;

b) the lymphoma can be a Hodgkin lymphoma or a non-Hodgkin lymphoma;

c) the administering can result in a reduced number of TSPAN33+ B-cells in the patient;

d) the anti-TSPAN33 antibody can be a monoclonal antibody, a neutralizing antibody, or a humanized antibody, or a combination thereof; or

e) a combination of a-d.

In another aspect, a method of treating an immune disease in which TSPAN33 is upregulated is provided. The method includes administering an anti-TSPAN33 antibody to a patient in need of such treatment in an amount effective to treat the immune disease.

In the method:

a) the immune disease can be an allergy or an autoimmune disease;

b) the disease can be rheumatoid arthritis, psoriasis, atopic dermatitis, Sjogren's syndrome, autoimmune hepatitis, primary biliary cirrhosis, ulcerative colitis, Crohn's disease, scleroderma, hypersensitivity pneumonitis, autoimmune thyroditis, hashimoto thyroiditis, Graves' disease, ankylosing spondylitis, Celiac disease, idiopathic thrombocytopenic purpura, mixed connective tissue disease, multiple sclerosis, multiple myeloma, pemphigus vulgaris, temporal arteritis, vitiligo, or systemic lupus erythematosus;

c) the disease can be rheumatoid arthritis or systemic lupus erythematosus;

d) the administering can result in a reduced number of TSPAN33+ B-cells in the patient;

e) the anti-TSPAN33 antibody can be a monoclonal antibody, a neutralizing antibody, or a humanized antibody, or a combination thereof; or

f) any combination of a-e.

In a further aspect, a method of purifying activated B-lymphocytes is provided. The method includes mixing an anti-TSPAN33 antibody with a lymphocyte-containing cell preparation, and separating lymphocytes bound by the antibody. In the method, the anti-TSPAN33 antibody can be a monoclonal antibody, a neutralizing antibody, or a humanized antibody, or a combination thereof; and/or the separating can be by fluorescence-activated cell sorting.

In another aspect, a method of identifying an activated and/or diseased B-lymphocyte is provided. The method includes detecting upregulated expression of TSPAN33 in the lymphocyte.

In the method:

a) the detecting can include: adding an anti-TSPAN33 antibody to a sample comprising proteins of the lymphocyte; forming an immune complex between the antibody and TSPAN33 when TSPAN33 is present in the sample; and detecting the immune complex;

b) the detecting can include: preparing cDNA from RNA of the lymphocyte; amplifying the cDNA with primers specific for nucleotide sequences in the TSPAN33 gene, or hybridizing the cDNA to nucleotide sequences of the TSPAN33 gene; and detecting amplified products of the amplification reaction or detecting hybrids between the cDNA and the TSPAN33 nucleotide sequences;

c) the lymphocyte can be from a patient, and the method can further include administering an anti-TSPAN33 antibody to the patient when upregulated expression of TSPAN33 is detected; or

d) any combination of a) and c), or b) and c).

In another aspect, a method of diagnosing a lymphoma or immune disease involving activated and/or diseased B-lymphocytes is provided. The method includes analyzing a sample of a patient for the presence of an activated and/or diseased B-lymphocyte by detecting upregulated expression of TSPAN33 in a lymphocyte of the sample, the patient being diagnosed with the lymphoma or immune disease when the activated and/or diseased B-lymphocyte is detected.

In the method:

a) the disease can be Hodgkin lymphoma, a non-Hodgkin lymphoma, precursor T-cell leukemia/lymphoma, follicular lymphoma, diffuse large B cell lymphoma, mantle cell lymphoma, B-cell chronic lymphocytic leukemia/lymphoma, MALT lymphoma, Burkitt's lymphoma, Burkitt's lymphoma, peripheral T-cell lymphoma-Not-Otherwise-Specified, nodular sclerosis form of Hodgkin lymphoma, or mixed-cellularity subtype of Hodgkin lymphoma;

b) the disease can be rheumatoid arthritis, psoriasis, atopic dermatitis, Sjogren's syndrome, autoimmune hepatitis, primary biliary cirrhosis, ulcerative colitis, Crohn's disease, scleroderma, hypersensitivity pneumonitis, autoimmune thyroditis, hashimoto thyroiditis, Graves' disease, ankylosing spondylitis, Celiac disease, idiopathic thrombocytopenic purpura, mixed connective tissue disease, multiple sclerosis, multiple myeloma, pemphigus vulgaris, temporal arteritis, vitiligo, or systemic lupus erythematosus;

c) the disease can be Hodgkin lymphoma, a non-Hodgkin lymphoma, rheumatoid arthritis or systemic lupus erythematosus;

d) detecting upregulated expression of TSPAN33 in a lymphocyte of the sample can be by any method of detecting upregulated expression of TSPAN33 described herein.

### BRIEF DESCRIPTION OF THE DRAWINGS

For a more complete understanding of the present invention, reference is now made to the following descriptions taken in conjunction with the accompanying drawings, in which:

FIG. 1 is the amino acid sequence of human TSPAN33 (SEQ ID NO:1).

FIG. 2 is an amino acid sequence comparison of human TSPAN 33 (SEQ ID NO:1) and mouse TSPAN33 (SEQ ID NO:2). A consensus sequence (SEQ ID NO:3) is also shown.

FIG. 3 is a graph showing that TSPAN33 expression is restricted to activated B cells in normal human tissues. Affymetrix gene array (U133 plus 2.0) data compiled from the human body index of gene expression database observing TSPAN33 expression in normal human tissue (n=8) and immune cells. X axis is organized by organ systems: CNS (central nervous system), Gut (gastrointestinal), Struct (structural), Vasc (vasculature), Resp (respiratory), Endo (endocrine), Ur (urinary), Rep (reproductive), Imm_T (immune tissue), Imm_C (immune cells), and Dev (developmental).

FIG. 4 is a panel showing that TSPAN33 expression is restricted to activated B cells in mice and humans. 4A) qRT-PCR of TSPAN33 expression in resting and activated (anti-CD40+IL-4) human B lymphocytes purified from human blood compared to human bone marrow, n=3. 4B) Western blot of PBMC's for TSPAN33 expression under resting and activating conditions with CpG+pokeweed mitogen (PWM)+pansorbin using actin as a loading control. Also shown are densitometric analyses. 4C) qRT-PCR of TSPAN33 expression over time in human 2E2 B cells (Black bars) with anti-CD40 mAb+IL-4 stimulation and human Jurkat T cells (White bars) under unstimulated, anti-CD3+anti-CD28 mAb, or PMA+ionomycin stimulation for 12 hrs, n=3. 4D) Western blot of TSPAN33 expression in resting vs. activated human 2E2 B cells with anti-CD40 mAb+IL-4. Also shown are densitometric analyses. 4E) qRT-PCR of Tspan33 A20-2J B cells under resting and activating conditions with 0.1, 1, or 10 ng/mL of LPS+IL-4. 4F) qRT-PCR of resting or stimulated B cells enriched from C57BL/6 spleens with 10 ng/mL LPS+IL-4 for 12 hours, n=3,* p<0.05, ** p<0.01 and *** p<0.001 indicate statistical significance according to Student's t test. Data are representative of three independent experiments. Error bars indicate standard deviation (SD).

FIG. 5 is a panel showing that TSPAN33 is expressed in human Hodgkin's and non-Hodgkin's lymphoma. 5A) qRT-PCR was performed on several human NHL lines and measured for TSPAN33 (Black bars) vs. MS4A1/CD20 (White bars) expression. Samples were normalized to GAPDH. 5B) RT-PCR expression analysis corresponding to the large extracellular loop 33 (LEL) of TSPAN33 in human Burkitt's lymphoma lines Raji, Ramos, and Daudi against BaF3 (a mouse pro-B cell line) compared to GAPDH. 5C) Western blot analysis of TSPAN33 expression of Raji, Ramos, Daudi, and BaF3 cells using a rabbit anti-TSPAN33 polyclonal antibody. Data are representative of three independent experiments.

FIG. 6 is a panel of images showing that TSPAN33 is expressed in human lymphomas. Lymphoma biopsies were sectioned and stained with Hematoxilin/Eosin and anti-TSPAN33, followed by isotype or anti-rabbit IgG-HRP. White arrows indicate Reed-Stenberg cells and black arrows indicate positive TSPAN33-stained cells. Representative images from biopsies taken from patients diagnosed HL (n=6), DLBCL (n=6), and mantle cell lymphoma (n=2).

FIG. 7 is a panel showing that TSPAN33 is upregulated in B cell-associated autoimmunity. 7A) qRT-PCR of Tspan33 expression of total splenocytes taken from MRL/faslpr/lpr mice normalized to CD19 expression. Mice ages 9 weeks old (no detectable pathology), 24 weeks old (lymphadenopathy with or without mild ear lesions) and 36 weeks old (lymphadenopathy with ear and face lesions) were compared for Tspan33 expression, n=5. 7B) qRT-PCR of Tspan33 expression in CD19+CD138− and CD19−CD138+ splenocytes from 11.5 week old female (lymphadenopathy) and 12.5 week old male (no pathology) MRL/faslpr/lpr mice, n=2. 7C) qRT-PCR of TSPAN33 expression analysis of PBMCs from human SLE patients or healthy controls, n=9. 7D) Microarray analysis of TSPAN33 vs MS4A1/CD20 expression in synovial membranes from healthy and RA patients. Synovial membranes were isolated from controls or RA patients as described (H. Soto, P. Hevezi, R. B. Roth, A. Pahuja, D. Alleva, H. M. Acosta, C. Martinez, A. Ortega, A. Lopez, R. Araiza-Casillas, A. Zlotnik, Gene array analysis comparison between rat collagen-induced arthritis and human rheumatoid arthritis, Scand J Immunol, 68 (2008) 43-57). The RNA was isolated from the membranes and analyzed for MS4A1/CD20 and TSPAN33 expression using the Affymetrix gene array U133 plus 2.0, n=9 healthy and n=5 RA patients, * p<0.05, ** p<0.01, ***p<0.001 (Student's t test). Data are representative of at least three independent experiments (A-C). Error bars indicate standard deviation (SD).

FIG. 8 is a panel of images showing that TSPAN33 is expressed in the proximal, distal convoluted tubules and collecting duct but not in the kidney glomerulus. Kidney biopsies were stained for IHC in a tissue array. Samples were stained with H&E and anti-TSPAN33 or rabbit IgG isotype control, followed by anti-rabbit IgG-HRP. 8A) 40× magnification showing Lymphocytes (black arrows) and nerves (white arrows). 8B) 40× magnification showing proximal convoluted tubules (black arrows) and kidney glomeruli (white arrows). 8C) 40× magnification showing distal convoluted tubule (black arrows) and collecting duct (white arrows). 8D) 100× magnification of proximal convoluted tubule showing the apical surface (black arrow) and granules (white arrows).

### DETAILED DESCRIPTION

Priority is claimed to U.S. Provisional Application No. 61/740,946, filed on Dec. 21, 2012, and which is incorporated by reference herein.

Tetraspanin 33 is a member of the tetraspanin family of membrane proteins (16) and was mapped to human chromosome 7 (7q31.2-q32) (17), a region that is a hotspot for deletions in myelodysplastic syndromes and acute myelogenous leukemias (17) Tetraspanin 33 was first characterized as a new tetraspanin involved in erythropoiesis (17-18). Tetraspanin 33 was also named Penumbra, Pen, (17), for Proerythroblast nu (new) membrane, as mice with a targeted deletion of the Pen gene (Pen^{-/-}) developed abnormal larger basophilic RBCs with anemia and splenomegaly (18). Penumbra expression was found highest in the bone marrow of the mouse, among the TER119⁺ fraction that includes all erythroblasts, while in neutrophils, resting T cells, resting B cells, monocytes, or natural killer cells Penumbra expression was low or undetectable (18). Although the latter study found that the TER119+ B cells were the highest Tspan 33-expressing cells of the bone marrow, our own data included herein indicate that tetraspanin 33 expression in activated B cells is 40-fold higher than in total bone marrow. The latter observation leads us to conclude that activated B cells represent cells with the highest expression of Tspan33/BAAM in the human body. This makes Tspan33/BAAM a unique candidate as a target of therapeutic antibody development to treat lymphomas or certain human autoimmune diseases where B cells are involved in their pathogenesis.

Human tetraspanin 33 (encoded by TSPAN33) has been identified as a biomarker found on B cell lymphomas using a comprehensive database of gene expression profiles (body index of gene expression) of over 90 different tissue and organs (19). Human tetraspanin 33 is a member of the transmembrane 4 superfamily with 97% homology to murine tetraspanin 33 and is involved in hematopoiesis (18). The high level of conservation between mouse and human BAAM genes makes mouse models suitable for preclinical studies that involve antibody targeted therapy.

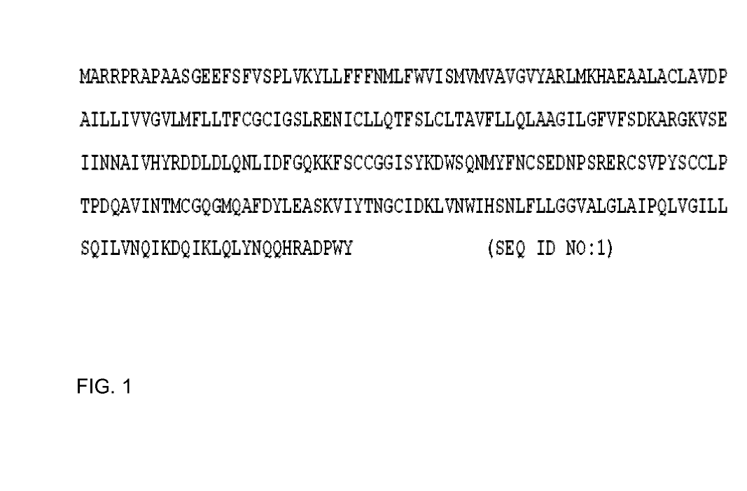

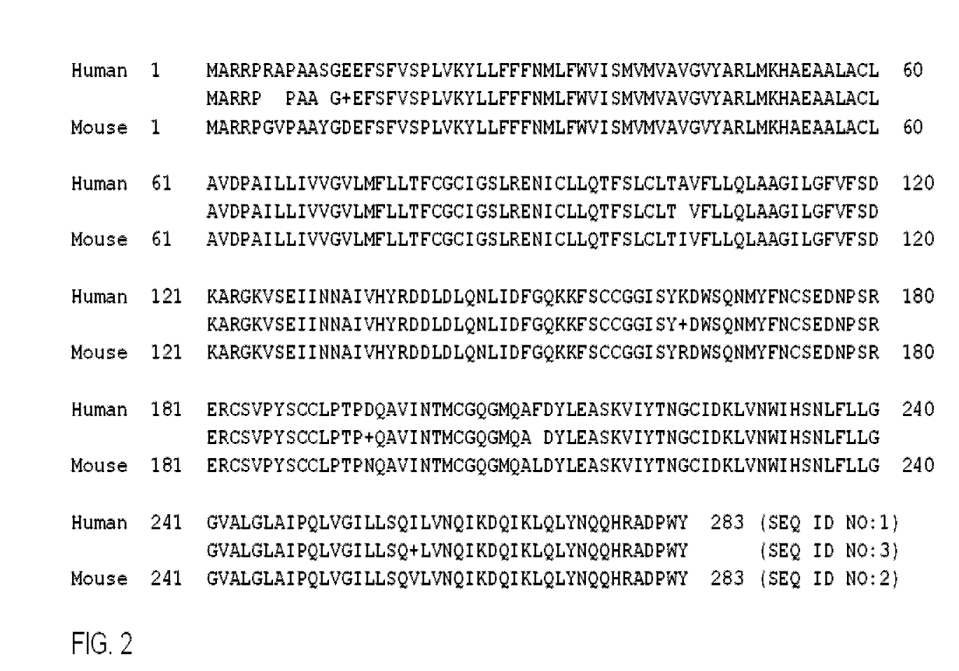

The human tetraspanin 33 protein sequence is provided in FIG. 1. A human vs. mouse TSPAN33 protein alignment is shown in FIG. 2. The human TSPAN33 nucleotide sequence accession number is NM 178562 (incorporated by reference herein), while the human TSPAN33 protein sequence accession number is NP_848657 (incorporated by reference herein).

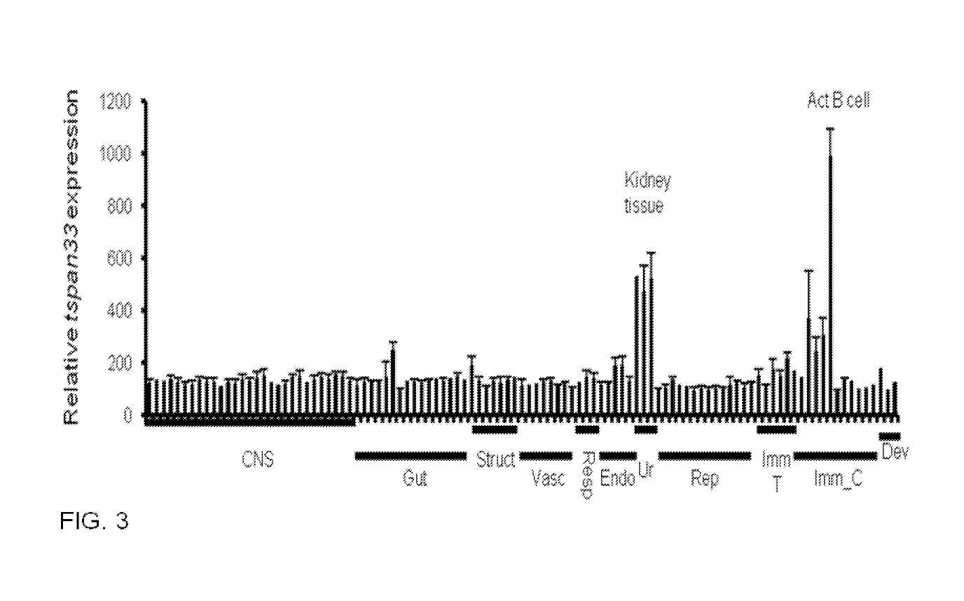

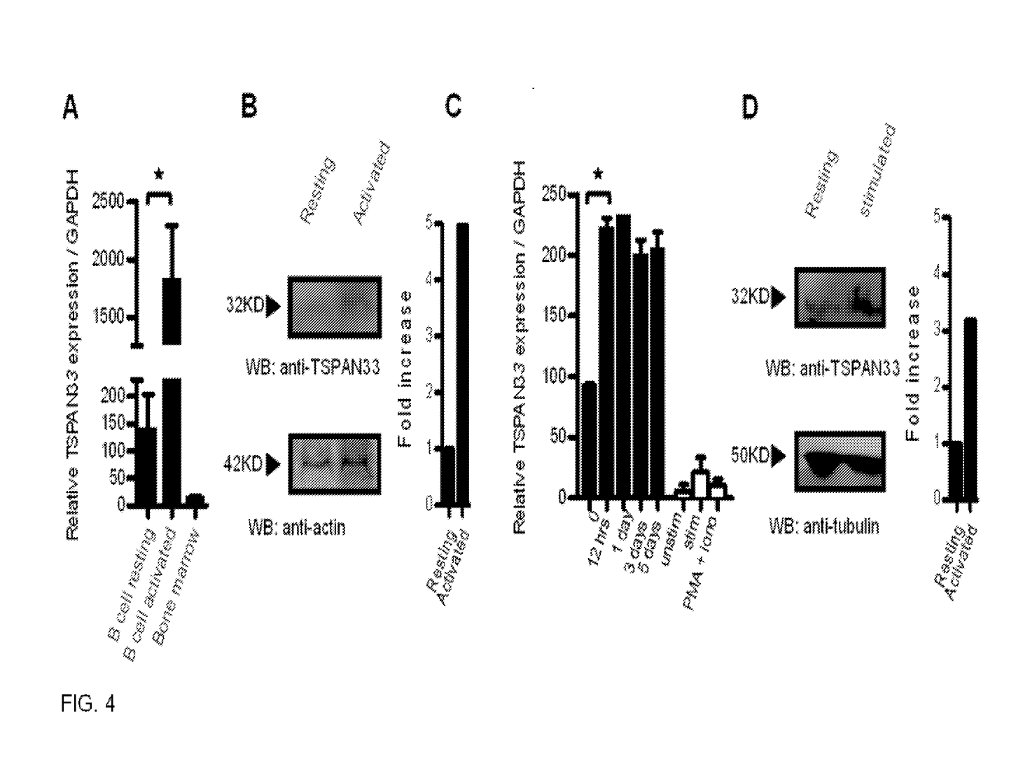

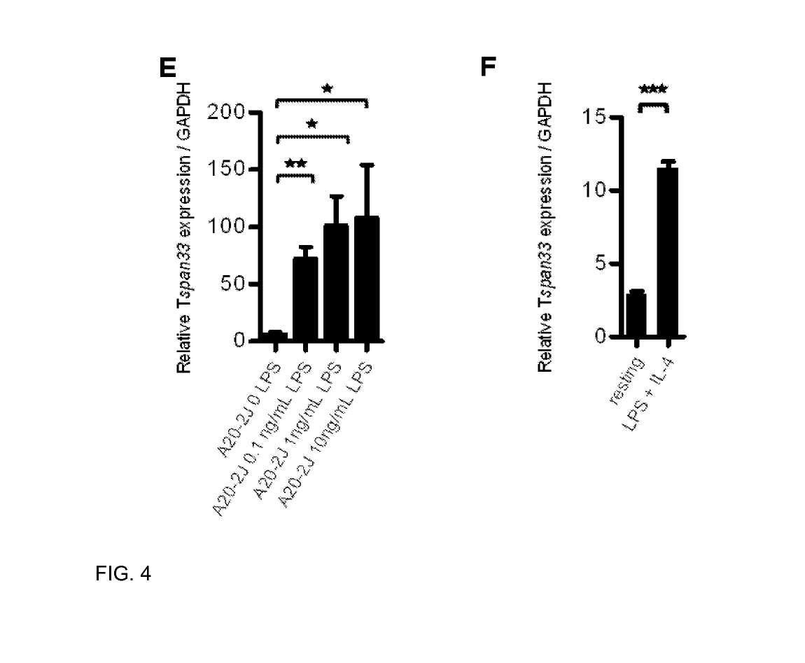

A comprehensive database of gene expression (Body Index of Gene Expression: BIGE (19)) has been used to map the expression of Tspan33 in 105 tissues and cells of the human body. The BIGE database indicates that the expression of Tspan 33 is highly specific and the highest levels of expression are in activated B cells (FIG. 3). The inventors therefore decided to rename this molecule BAAM, or B cell Activation Associated Molecule, a name that better reflects its expression pattern in humans. Another site with significant levels of BAAM expression is the kidney (FIG. 3). This pattern of BAAM expression was confirmed using qRT-PCR of human RNAs (FIG. 4) with high levels of BAAM mRNA detected in the kidney. All other tissues including primary (bone marrow and thymus) and secondary lymph organs (spleen), as well as resting B cells were negative for BAAM expression.

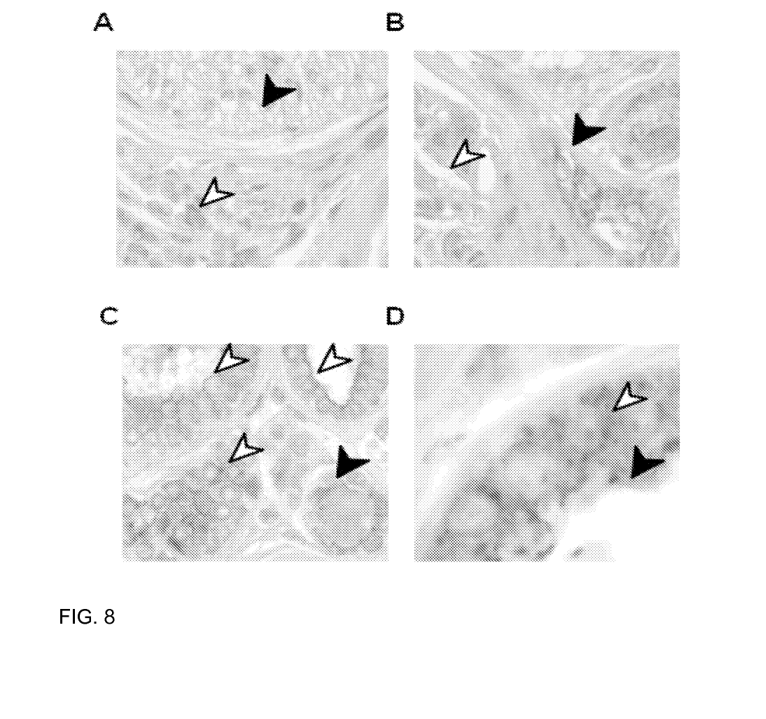

Among the extra-lymphoid sites of BAAM expression, the expression in kidneys raised concerns for the possible therapeutic uses of anti-BAAM antibodies in vivo. To assess the potential of offsite targeting of therapeutic monoclonal antibodies against BAAM in the kidneys, immunohistochemistry was performed using anti-BAAM polyclonal antibodies (FIG. 8). These results revealed that BAAM is expressed in the proximal and distal convoluted tubules of the kidney, while lymphocytes, nerves, kidney collecting duct and glomeruli did not express BAAM. The proximal and distal convoluted tubules are lined with epithelial brush border cells that are involved in secretion and absorption of proteins, ions, and organic solutes during urine filtration. Expression of BAAM in the kidney is therefore unrelated to B cell activation and probably involved in vesicular trafficking or signaling in these cells, since tetraspanins, as a family, have been linked to these functions (16). Importantly, only low molecular weight proteins can cross from the afferent vessels of the blood stream through the glomerular space and enter the convoluted tubules where the urine will be collected for transfer to the bladder. Therefore, therapeutic monoclonal antibodies targeted to BAAM should not reach their target and affect kidney function, as antibodies do not enter this space. In addition, kidney epithelial cells have been reported to be refractory towards biological based cytotoxic agents and kidney cell carcinomas are also reported to be resistant to ADCC (20). Taken together, these data indicate that Tspan33/BAAM kidney expression should not raise concerns for the therapeutic use of anti-BAAM antibodies in humans.

Since TSPAN 33 is highly conserved (21) and highly upregulated in activated B cells, it is expected to participate in the activation of B cells. Therefore, in an embodiment, an antibody is used to regulate B cell activation and treat autoimmune or allergic immune diseases. The term “upregulated expression” means the expression is increased compared to a control. For example, expression of TSPAN33 can be increased relative to a control gene, or expression can be increased relative to the expression in a control cell.

B cell activation markers are important as diagnostic tools as elevated levels of B cell activation markers have been shown to be associated with cancer risk such as Non-Hodgkin Lymphoma (NHL) (22-23). To this end, the inventors reasoned that BAAM would be expressed in human lymphomas because other B cell antigens (notably CD19 and CD20) are also highly expressed in these tumors (24). To assess the expression of TSPAN33 in NHL, RT-PCR was performed on several diffuse B cell lymphomas (non-Hodgkin lymphoma) and the results indicate that BAAM expression was comparable to CD20 expression. RT-PCR and western blotting was also performed on several human Burkitt's lymphoma cell lines (non-Hodgkin lymphoma) and TSPAN33 was readily detected at both the mRNA and protein levels. Furthermore, immunohistochemistry was performed on biopsies from patients with aggressive NHL, mantle cell lymphoma (NHL), and Hodgkin lymphoma containing Reed-Sternberg cells. The results indicate that the latter are highly positive for BAAM. The mantle cell lymphoma was negative for BAAM. BAAM expression could be related to the activation state of the B cell lymphoma. Reed-Sternberg cells are thought to be derived from germinal center B cells that have acquired disadvantageous somatic hypermutation and failed to undergo apoptosis, and therefore they are an activated form of lymphoma (25). Mantle cell lymphoma, on the other hand, are a type of mature CD5+ B cell lymphoma containing a translocation of the cyclin-Dl gene on 11q13 to the promoter of the immunoglobulin heavy chain locus on 14q32 (26). The cells are thought to originate from naïve, pre-germinal center lymphocytes, thus are a form of non-activated B lymphocytes (26). Thus, the differences in usefulness of TSPAN33 as a target of therapeutic antibodies towards lymphomas could be related to their activation state.

Markers of B cell activation are also associated with certain autoimmune diseases. For instance, serum immunoglobulin, IL-6 and IL-21 levels are all significantly elevated in patients newly diagnosed with Rheumatoid Arthritis (RA) (27-28). To further explore the role of activated B cells in RA, and expression levels of BAAM as a potential biomarker for RA, microarray data was used from a global gene expression analysis of synovial membranes of 9 normal and 5 RA patients undergoing reconstructive, or, replacement knee surgery respectively (29). Levels of both BAAM (p=0.0019) and CD20 (p=0.0008) mRNAs were elevated in the samples obtained from patients with Rheumatoid Arthritis. In addition, the top 25 probe sets elevated in the RA samples represent markers of B cell activation, including immunoglobulin light and heavy chain genes, which is consistent with the role of activated B cells in RA (29). BAAM is concluded to be a biomarker for activated B cells found in RA lesions in humans. These data indicate that anti-BAAM antibodies would eliminate activated B cells from these lesions and therefore would ameliorate the condition in RA patients. These observations are expanded to other autoimmune diseases where activated B cells are involved, including (but not restricted to) psoriasis, atopic dermatitis, Sjogren's syndrome, autoimmune hepatitis, primary biliary cirrhosis, ulcerative colitis, Crohn's disease, scleroderma, hypersensitivity pneumonitis, autoimmune thyroditis, hashimoto thyroiditis, Graves' disease, ankylosing spondylitis, Celiac disease, idiopathic thrombocytopenic purpura, mixed connective tissue disease, multiple sclerosis, multiple myeloma, pemphigus vulgaris, temporal arteritis, vitiligo, and systemic lupus erythematosus.

Some embodiments of the present invention are based on the findings that BAAM is a marker of activated B cells and certain types of lymphomas. In one aspect, the present invention provides new and specific uses of therapeutic antibodies to treat diseases such as types of BAAM positive lymphomas and leukemias, as well as autoimmune diseases involving activated B cells. In another aspect, the present invention provides the use of BAAM as a biomarker of B cell activation for the diagnosis of allergies, autoimmune diseases, or lymphomas involving the presence of this protein. Thus, some embodiments of the present invention provide new and specific uses for a therapeutic antibody against TSPAN33, produced by one skilled in the art, as a target to treat TSPAN33 positive lymphomas or autoimmune diseases involving activated B cells. Also, some embodiments of the present invention provide for the use of TSPAN33 as a biomarker of activated B cells, to be used in diagnosis of diseases involving activated B cells, such as TSPAN33 positive lymphomas, autoimmune diseases, or allergies.

Some embodiments are based on the identification and characterization of TSPAN33/BAAM and the finding that it is upregulated in activated B lymphocytes and certain lymphomas. These embodiments provide new and specific uses of therapeutic monoclonal antibodies “loaded” or “naked,” to treat any diseases involving lymphomas or autoimmune disorders that are TSPAN33/BAAM positive. The words “loaded” and “naked” refers to whether or not the antibody is conjugated to a cytotoxic agent, such as radioactive agent, free radical, or toxin, in which the antibody would be known as loaded. The word “naked” refers to a therapeutic antibody that is not conjugated to a cytotoxic agent. It is well understood in the art that conjugating a cytotoxic agent could potentially improve the therapeutic use of monoclonal antibodies, by increasing the “potency” of the antibody through the delivery of a cytotoxic agent to a specific target using the antibody as a homing missile.

An anti-TSPAN33 antibody can target activated and/or diseased B lymphocytes expressing TSPAN33 and lead to their depletion via complement mediated cytoxicity (CMC) or antibody dependent cellular cytotoxicity (ADCC), or more directly by altering cell behavior. In addition, an anti-TSPAN33 antibody can be used an antibody-drug conjugate to increase the killing ability of the antibody against cells expressing TSPAN33. The use of antibodies to deplete B cells has been shown to be an effective therapy, for example, as with the anti-CD20 monoclonal antibody Rituximab.

Monoclonal antibodies produced from mouse cells require humanization in order to be used in humans. There are several ways of doing this. One is by producing humanized antibodies where the mouse regions of the antibody (crystallizable fragment or Fc) are replaced with human Fc sequences. This can be done in a variety of ways using molecular biology techniques by persons skilled in the art (7-8). Alternatively, the antibodies can be produced by immunizing mice that have had their immune system changed from mouse to human by using molecular biology techniques. Several such mice have been produced (7). In certain embodiments, new and specific uses of humanized or fully human monoclonal antibodies are produced through these known methods, loaded or naked, towards TSPAN33/BAAM as a target for therapeutic antibodies to treat any diseases involving TSPAN33/BAAM positive diseased B cells.

The treating of any disease involving TSPAN33/BAAM positive diseased B cells is based on the findings that TSPAN33/BAAM is determined to be a biomarker of activated B cells and certain types of lymphomas. The diseased B cells are contemplated to extend to allergic immune related diseases and autoimmune diseases involving TSPAN33/BAAM positive diseased B cells, such as in antibodies produced to allergens and Rheumatoid arthritis, respectively.

In one embodiment is provided a method of treating any lymphoma or leukemia that is TSPAN33/BAAM -positive by using the biomarker as a target for therapeutic monoclonal antibodies. This includes any lymphoma type such as Hodgkin lymphoma or the variety of non-Hodgkin lymphomas that express this molecule, including certain T cell lymphomas that may express TSPAN33/B AAM. Other lymphomas for treatment include: Precursor T-cell leukemia/lymphoma; Follicular lymphoma; Diffuse large B cell lymphoma; Mantle cell lymphoma; B-cell chronic lymphocytic leukemia/lymphoma; MALT lymphoma; Burkitt's lymphoma; Burkitt's lymphoma; Peripheral T-cell lymphoma-Not-Otherwise-Specified; Nodular sclerosis form of Hodgkin lymphoma; Mixed-cellularity subtype of Hodgkin lymphoma. In another embodiment is provided a method for treating any immune disease containing diseased B lymphocytes that express the biomarker, including allergies and autoimmune diseases. Hypersensitive allergic B lymphocytes that possess antibodies towards allergens can be depleted using the TSPAN33 as a target for therapeutic antibodies. Likewise, autoreactive B lymphocytes that possess autoantibodies to self antigens could similarly be depleted, using any method mentioned earlier.

In another embodiment is provided a means to regulate B cell activation or presentation to T cells by blocking TSPAN33/BAAM using a neutralizing antibody. This is based on the finding that TSAN33/BAAM is over 97% conserved in humans and mice, thus may have a role in B cell function, activation, proliferation, or trafficking. Therefore developing a neutralizing antibody by one who is skilled in the art, could be used to block B cell function. This could also be used to modulate the immune response of humoral immunity to treat a variety of diseases, such as allergies or autoimmunity by inhibiting B cell activation or presentation if TSPAN33/BAAM does in fact play a role in this function. A neutralizing antibody can be screened using an assay in which the antibody binds to the large extracellular loop (LEL) region of the TSPAN33 molecule. For example, soluble LEL can be expressed by cloning the nucleotide sequence corresponding to the LEL portion of TSPAN33 into an expression vector, which is then transfected into an appropriate host cell. The ability of an anti-TSPAN33 antibody to bind LEL can be assayed by Western blot.

An antibody is an immunologic binding agent such as IgG, IgM, IgA, IgD and IgE. Techniques for preparing and using various antibody-based constructs and fragments are well known in the art. Means for preparing and characterizing antibodies are also well known in the art (See, for example, Harlow and Lane, “Antibodies: A Laboratory Manual,” Cold Spring Harbor Laboratory, 1988, incorporated by reference herein). Monoclonal antibodies (mAbs) are recognized to have certain advantages, e.g., reproducibility and large-scale production. Thus, monoclonal antibodies of the human, murine, monkey, rat, hamster, rabbit and even chicken origin, are contemplated for use. In some embodiments, an antibody-like molecule that has an antigen binding region may be appropriate. Examples of such anti-body like molecules include, but are not limited to, antibody fragments such as Fab′, Fab, F(ab′)₂, single domain antibodies (DABs), Fv, scFv (single chain Fv), and the like.

Polyclonal antibodies can be prepared in a wide range of animal species. Typically, the animal used for production of antisera is a rabbit, a mouse, a rat, a hamster, a guinea pig or a goat. To increase immunogenicity, use of adjuvants and conjugation to a carrier protein such as, but not limited to, keyhole limpet hemocyanin or bovine serum albumin are well known procedures.

A monoclonal antibody can be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain. The immunizing composition is administered in a manner effective to stimulate antibody producing cells (31-33).

For example, following several immunizations, the presence of anti-TSPAN33 antibodies in the serum of the mouse can be assayed by testing the serum by enzyme-linked immunosorbant assay (ELISA). Once the presence of anti-TSPAN33 antibodies is confirmed in the serum of a given mouse, its spleen can be fused to a myeloma cell suitable for the production of monoclonal antibodies using several techniques like PEG-driven fusion or electrical techniques. The resulting hybridomas can be selected in HAT medium and screened for the production of anti-TSPAN33 antibodies by ELISA.

A polyclonal or monoclonal antibody can be further purified, if desired, using filtration, centrifugation and various chromatographic methods such as HPLC or affinity chromatography.

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions. Thus, humanized antibodies against TSPAN33 are included in some embodiments, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art (34-39). For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

Different formulations or pharmaceutical compositions (sterile, buffered, slow release, controlled release, stabilizers, ointments, etc.) of an antibody can be used for therapeutic treatment depending on the optimal route of administration. See, e.g., Niazi S. K. Handbook of Pharmaceutical Manufacturing Formulations Informa Healthcare 2012. In addition, the compound(s) can be used in combination with other therapeutics in a single formulation strategy. Pharmacological variants can be used to obtain desired pharmacokinetic outcomes (secretion, half life, solubility or optimize excretion routes).

The exact dose of the antibody will depend on the purpose of the treatment, and will be ascertainable by one skilled in the art using known techniques. See, e.g., Ansel, et al., Pharmaceutical Dosage Forms and Drug Delivery; Lieberman (1992) Pharmaceutical Dosage Forms (vols. 1-3), Dekker, ISBN 0824770846, 082476918X, 0824712692, 0824716981; Lloyd (1999) The Art, Science and Technology of Pharmaceutical Compounding; and Pickar (1999). As is known in the art, adjustments for protein degradation, systemic versus localized delivery, and rate of new protease synthesis, as well as the age, body weight, general health, sex, diet, time of administration, drug interaction, and the severity of the condition may be necessary, and will be ascertainable with some experimentation by those skilled in the art.

Various pharmaceutically acceptable excipients are well known in the art and can be included in a formulation or pharmaceutical composition. As used herein, “pharmaceutically acceptable excipient” includes a material which, when combined with an active ingredient of a composition, allows the ingredient to retain biological activity and without causing disruptive reactions with the subject's immune system. Such may include stabilizers, preservatives, salt or sugar complexes or crystals, and the like. See, e.g., Niazi S. K. Handbook of Pharmaceutical Manufacturing Formulations Informa Healthcare 2012.

Exemplary pharmaceutically acceptable carriers that can be included in a formulation or pharmaceutical composition include sterile aqueous or non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In other embodiments, the compositions will be incorporated into solid matrix, including slow release particles, glass beads, bandages, inserts on the eye, and topical forms. Administration routes may include the following: topical, systemic, intravenous, intraperitoneal, respiratory, oral, eye, implant, vaginal, anal, suppository, devices with control release, etc.

Existing therapeutics for the indications described elsewhere in this application can be used in combination or sequentially with anti-TSPANN33 antibody to optimize therapeutic outcomes.

Another embodiment provides a means to screen for diseased B lymphocytes using assays that detect the presence of this biomarker. Examples include, but are not limited to, ELISA, polymerase chain reaction (PCR), or fluorescence-activated cell sorting (FACS) assays that can be used to screen for the expression of TSPAN33/BAAM as a biomarker of activated B lymphocytes or diseased B lymphocytes. Above “normal” levels of TSPAN33/BAAM expression could indicate lymphoma or a hyperactive immune response, such as seen in allergies.

Immunodetection methods for detecting TSPAN33 can include ELISA, radioimmunoassay (RiA), fluoroimmunoassay, chemiluminescent assay, bioluminescent assay, Western blotting, and immunohistochemistry. In these methods, a sample is contacted with a first antibody that has affinity for the target protein to form immune complexes, and then the immune complexes are detected, for example, by a label attached to the first antibody (such as a radioactive, fluorescent or enzyme label), or by means of a secondary binding molecule (such as a second antibody) that has affinity for the first antibody. The secondary molecule can be linked to a label for detection.

Nucleic acid detection methods include PCR-based and hybridization-based methods. PCR-based methods include, but are not limited to, reverse transcription PCR (RT-PCR), reverse transcription quantitative PCR (RT-qPCR), or standard PCR. In PCR-based methods, RNA from a cell or tissue sample is reverse transcribed into cDNA, then amplified using primers. Examples of hybridization-based methods include, but are not limited to, DNA microarrays, Northern blotting, and in situ hybridization. In hybridization-based methods, RNA from a cell or tissue sample is reverse transcribed into labeled cDNA (fluorescently labeled, for example), which is then used to probe, for example, DNA microarrays, Northern blots, or tissue sections prepared for in situ hybridization.

In another embodiment, this invention provides a means to sort or purify activated B lymphocytes using cell separation, purification columns, or FACS sorting using the biomarker TSPAN33/BAAM as a marker of activated B lymphocytes

### Uses of Antibody Targeted Therapy Towards TSPAN33/BAAM to Treat Disease

In some embodiments, antibody targeted therapy towards TSPAN33/BAAM can be used as a treatment for TSPAN33/BAAM positive lymphomas. For example, biopsies were taken from patients with mantle cell lymphoma (NHL), aggressive non-Hodgkin lymphoma, and Reed-Sternberg cell containing Hodgkin lymphomas. The tissue were sectioned and stained for TSPAN33 using an HRP conjugated anti-mouse IgG against the H&E stain. The Hodgkin lymphoma and aggressive non-Hodgkin lymphomas are thought to be derived from activated B lymphocytes (25), while mantle cell lymphomas are thought to be derived from naïve, pre-germinal center B lymphocytes (26), thus represent a form of non-activated B lymphoma. Only the Hodgkin and aggressive non-Hodgkin lymphoma sections were positive for TSPAN33. Thus TSPAN33/BAAM are contemplated to be an effective target for therapeutic monoclonal antibodies in TSPAN33/BAAM positive lymphomas.

In some embodiments, antibody targeted therapy towards TSPAN33/BAAM can be used to treat autoimmune diseases involving TSPAN33/BAAM positive and autoantibody secreting, B lymphocytes. Thus provided is the treatment of autoimmune diseases involving TSPAN33/BAAM positive and autoantibody producing autoimmune diseases, that includes, but is not limited to, Rheumatoid Arthritis, psoriasis, Sjogren's syndrome and Lupus Erythematosus.

In some embodiments, neutralizing antibodies towards TSPAN33/BAAM can be used to treat immune diseases involving diseased B lymphocytes. These embodiments are based on the findings that TSPAN33/BAAM is over 97% conserved in mice and humans. The tetraspanin family has a variety of functions including regulation of cell morphology, motility, invasion, fusion and signaling, in the brain, immune system, on tumors and elsewhere (30). Thus TSPAN33/BAAM may be involved in the signaling, activation, proliferation, or presentation of B cells or their signaling to T cells. Thus using neutralizing antibodies to block TSPAN33/BAAM signaling is contemplated to be used to modulate the immune response in a favorable manner to treat immune diseases involving B cell dysregulation.

### Uses of TSPAN33/BAAM as a Screening Tool

In some embodiments, TSPAN33/BAAM is used as a biomarker of activated and diseased B lymphocytes as a diagnostic test. These embodiments are based on the finding that TSPAN33/BAAM is negative in resting B cells, but transcription increases over 40 fold after activation with anti-CD40+IL-4 after 12 hour. For example, 10⁶ cells/mL of purified human B cells and 2E2 human B cell lines were stimulated with 0.1 ug/mL anti-CD40 (G28.5 mAb) and 4 ng/mL of IL-4. The cells were lysed and RNA was harvested using a Qiagen RNeasy kit. 500 ug was used to make cDNA with random hexamers using the QIAGEN—QuantiTect Rev. Transcription Kit. RT-qPCR was performed on the cell lysates using the Roche Lightcycler 480 system. Tspann33 primers were developed using the lightcycler primer design program with forward primer 5′-caacatgctcttctgggtga-3′ (SEQ ID NO: 4) and reverse primer 5′-attagccgagcgtagacacc-3′ (SEQ ID NO: 5) using the UPL primer #9. CD20 was amplified using forward primer 5′-aacaaaatctctactttgatggaactt-3′ (SEQ ID NO: 6) and reverse primer 5′-gcaaggcctactgctgagtt-3′ (SEQ ID NO: 7) with UPL primer #60. Expression was normalized using an average of 18S and GAPDH expression. Thus an antibody or protein that binds to TSPAN33/BAAM made by one skilled in the art, is contemplated to be used as a screening tool for activated B cells or diseased B cells using assays including, but not limited, to ELISAs, flow cytometry, or ELISPOT. Some embodiments also extend to the use of PCR based methods, such as RT-PCR, RT-qPCR, or PCR to detect TSPAN33 as a screening tool for the detection of activated B cells or diseased B cells

### Uses of TSPAN33/BAAM as a Sorting Tool to Isolate or Identify Diseased B Lymphocytes

In some embodiments, TSPAN33/BAAM is used as a biomarker of activated and diseased B lymphocytes in cell sorting. These embodiments are based on the examples in the current application that activated B cells express TSPAN33/BAAM. Thus an antibody or protein that binds to TSPAN33/BAAM is contemplated to be used in cell sorting, separation, or FACS analysis to purify or label cells.

The following references are referred to above, and are incorporated by reference herein:
(1) Kaminski, D. A., Wei, C., Qian, Y., Rosenberg, A. F. and Sanz, I., Advances in human B cell phenotypic profiling. Front Immunol 2012. 3: 302.
(2) Vardiman, J. and Hyjek, E., World health organization classification, evaluation, and genetics of the myeloproliferative neoplasm variants. Hematology Am Soc Hematol Educ Program 2011. 2011: 250-256.
(3) Went, P., Agostinelli, C., Gallamini, A., Piccaluga, P. P., Ascani, S., Sabattini, E., Bacci, F., Falini, B., Motta, T., Paulli, M., Artusi, T., Piccioli, M., Zinzani, P. L. and Pileri, S. A., Marker expression in peripheral T-cell lymphoma: a proposed clinical-pathologic prognostic score. J Clin Oncol 2006. 24: 2472-2479.
(4) Silverstein, A. M., The collected papers of Paul Ehrlich: why was volume 4 never published? Bull Hist Med 2002. 76: 335-339.
(5) Gaffar, S. A., Pant, K. D., Shochat, D., Bennett, S. J. and Goldenberg, D. M., Experimental studies of tumor radioimmunodetection using antibody mixtures against carcinoembryonic antigen (CEA) and colon-specific antigen-p (CSAp). Int J Cancer 1981. 27: 101-105.
(6) DeNardo, S. J., DeNardo, G. L., O'Grady, L. F., Hu, E., Sytsma, V. M., Mills, S. L., Levy, N. B., Macey, D. J., Miller, C. H. and Epstein, A. L., Treatment of B cell malignancies with 131I Lym-1 monoclonal antibodies. Int J Cancer Suppl 1988. 3: 96-101.
(7) Nelson, A. L., Dhimolea, E. and Reichert, J. M., Development trends for human monoclonal antibody therapeutics. Nat Rev Drug Discov 2010. 9: 767-774.
(8) Almagro, J. C. and Fransson, J., Humanization of antibodies. Front Biosci 2008. 13: 1619-1633.
(9) Presta, L. G., Molecular engineering and design of therapeutic antibodies. Curr Opin Immunol 2008. 20: 460-470.
(10) Sharkey, R. M. and Goldenberg, D. M., Targeted therapy of cancer: new prospects for antibodies and immunoconjugates. CA Cancer J Clin 2006. 56: 226-243.
(11) Robak, T. and Robak, E., New anti-CD20 monoclonal antibodies for the treatment of B-cell lymphoid malignancies. BioDrugs 2011. 25: 13-25.
(12) Mukohara, T., Role of HER2-Targeted Agents in Adjuvant Treatment for Breast Cancer. Chemother Res Pract 2011. 2011: 730360.
(13) Strickler, J. H. and Hurwitz, H. I., Bevacizumab-based therapies in the first-line treatment of metastatic colorectal cancer. Oncologist 2012. 17: 513-524.
(14) Smith, M. R., Rituximab (monoclonal anti-CD20 antibody): mechanisms of action and resistance. Oncogene 2003. 22: 7359-7368.
(15) Rehnberg, M., Amu, S., Tarkowski, A., Bokarewa, M. I. and Brisslert, M., Short- and long-term effects of anti-CD20 treatment on B cell ontogeny in bone marrow of patients with rheumatoid arthritis. Arthritis Res Ther 2009. 11: R123.
(16) Maecker, H. T., Todd, S. C. and Levy, S., The tetraspanin superfamily: molecular facilitators. FASEB J 1997. 11: 428-442.
(17) Chen, Z., Pasquini, M., Hong, B., DeHart, S., Heikens, M. and Tsai, S., The human Penumbra gene is mapped to a region on chromosome 7 frequently deleted in myeloid malignancies. Cancer Genet Cytogenet 2005. 162: 95-98.
(18) Heikens, M. J., Cao, T. M., Morita, C., Dehart, S. L. and Tsai, S., Penumbra encodes a novel tetraspanin that is highly expressed in erythroid progenitors and promotes effective erythropoiesis. Blood 2007. 109: 3244-3252.
(19) Roth, R. B., Hevezi, P., Lee, J., Willhite, D., Lechner, S. M., Foster, A. C. and Zlotnik, A., Gene expression analyses reveal molecular relationships among 20 regions of the human CNS. Neurogenetics 2006. 7: 67-80.
(20) Surfus, J. E., Hank, J. A., Oosterwijk, E., Welt, S., Lindstrom, M. J., Albertini, M. R., Schiller, J. H. and Sondel, P. M., Anti-renal-cell carcinoma chimeric antibody G250 facilitates antibody-dependent cellular cytotoxicity with in vitro and in vivo interleukin-2-activated effectors. J Immunother Emphasis Tumor Immunol 1996. 19: 184-191.
(21) Bradbury, A. R., Sidhu, S., Dubel, S. and McCafferty, J., Beyond natural antibodies: the power of in vitro display technologies. Nat Biotechnol 2011. 29: 245-254.
(22) De Roos, A. J., Mirick, D. K., Edlefsen, K. L., LaCroix, A. Z., Kopecky, K. J., Madeleine, M. M., Magpantay, L. and Martinez-Maza, O., Markers of B-cell activation in relation to risk of non-Hodgkin lymphoma. Cancer Res 2012. 72: 4733-4743.
(23) Breen, E. C., Hussain, S. K., Magpantay, L., Jacobson, L. P., Detels, R., Rabkin, C. S., Kaslow, R. A., Variakojis, D., Bream, J. H., Rinaldo, C. R., Ambinder, R. F. and Martinez-Maza, O., B-cell stimulatory cytokines and markers of immune activation are elevated several years prior to the diagnosis of systemic AIDS-associated non-Hodgkin B-cell lymphoma. Cancer Epidemiol Biomarkers Prev 2011. 20: 1303-1314.
(24) Anderson, K. C., Bates, M. P., Slaughenhoupt, B. L., Pinkus, G. S., Schlossman, S. F. and Nadler, L. M., Expression of human B cell-associated antigens on leukemias and lymphomas: a model of human B cell differentiation. Blood 1984. 63: 1424-1433.
(25) Kuppers, R., The biology of Hodgkin's lymphoma. Nat Rev Cancer 2009. 9: 15-27.
(26) Perez-Galan, P., Dreyling, M. and Wiestner, A., Mantle cell lymphoma: biology, pathogenesis, and the molecular basis of treatment in the genomic era. Blood 2011. 117: 26-38.
(27) Gottenberg, J. E., Miceli-Richard, C., Ducot, B., Goupille, P., Combe, B. and Mariette, X., Markers of B-lymphocyte activation are elevated in patients with early rheumatoid arthritis and correlated with disease activity in the ESPOIR cohort. Arthritis Res Ther 2009. 11: R114.
(28) Gottenberg, J. E., Dayer, J. M., Lukas, C., Ducot, B., Chiocchia, G., Cantagrel, A., Saraux, A., Roux-Lombard, P. and Mariette, X., Serum IL-6 and IL-21 are associated with markers of B cell activation and structural progression in early rheumatoid arthritis: results from the ESPOIR cohort. Ann Rheum Dis 2012. 71: 1243-1248.
(29) Soto, H., Hevezi, P., Roth, R. B., Pahuja, A., Alleva, D., Acosta, H. M., Martinez, C., Ortega, A., Lopez, A., Araiza-Casillas, R. and Zlotnik, A., Gene array analysis comparison between rat collagen-induced arthritis and human rheumatoid arthritis. Scand J Immunol 2008. 68: 43-57.
(30) Hemler, M. E., Tetraspanin functions and associated microdomains. Nat Rev Mol Cell Biol 2005. 6: 801-811.
(31) Butler M, Meneses-Acosta A (2012) Recent advances in technology supporting biopharmaceutical production from mammalian cells. Appl Microbiol Biotechnol 96: 885-894;
(32) Rasmussen S K, Naested H, Muller C, Tolstrup A B, Frandsen T P (2012) Recombinant antibody mixtures: production strategies and cost considerations. Arch Biochem Biophys 526: 139-145;
(33) ‘Marichal-Gallardo P A, Alvarez M M (2012) State-of-the-art in downstream processing of monoclonal antibodies: process trends in design and validation. Biotechnol Prog 28: 899-916, all incorporated by reference herein.
(34) Glassy M C (1993) Production methods for generating human monoclonal antibodies. Hum Antibodies Hybridomas 4: 154-165;
(35) Marichal-Gallardo P A, Alvarez M M (2012) State-of-the-art in downstream processing of monoclonal antibodies: process trends in design and validation. Biotechnol Prog 28: 899-916;
(36) Chon J H, Zarbis-Papastoitsis G (2011) Advances in the production and downstream processing of antibodies. N Biotechnol 28: 458-463;
(37) Di Fede G, Bronte G, Rizzo S, Rolfo Cervetto C, Cocorullo G, et al. (2011) Monoclonal antibodies and antibody fragments: state of the art and future perspectives in the treatment of non-haematological tumors. Expert Opin Biol Ther 11: 1433-1445;
(38) Chiarella P (2011) Production, novel assay development and clinical applications of monoclonal antibodies. Recent Pat Anticancer Drug Discov 6: 258-267;
(39) Kaneko E, Niwa R (2011) Optimizing therapeutic antibody function: progress with Fc domain engineering. BioDrugs 25: 1-11bb

The present invention may be better understood by referring to the accompanying examples, which are intended for illustration purposes only and should not in any sense be construed as limiting the scope of the invention.

### Example 1

We have identified Tspan33 as a gene encoding a transmembrane protein exhibiting a restricted expression pattern including expression in activated B cells. TSPAN33 is a member of the tetraspanin family. TSPAN33 is not expressed in resting B cells, but is strongly induced in primary human B cells following activation. Human 2E2 cells, a Burkitt's lymphoma-derived B cell model of activation and differentiation, also upregulate TSPAN33 upon activation. TSPAN33 is expressed in several lymphomas including Hodgkin's and Diffuse large B Cell Lymphoma. TSPAN33 is also expressed in some autoimmune diseases where B cells participate in the pathology, including rheumatoid arthritis patients, systemic lupus erythematosus (SLE), and in spleen B cells from MRL/Fas^{lpr/lpr} mice (a mouse model of SLE). We conclude that TSPAN33 may be used as a diagnostic biomarker or as a target for therapeutic antibodies for treatment of certain B cell lymphomas or autoimmune diseases.

Abbreviations used in the examples BCMA, B cell Maturation Antigen; BIGE, Body Index of Gene Expression (database); TSPAN33, tetraspanin 33; BL, Burkitt's lymphoma; RA, Rheumatoid arthritis; NHL, non-Hodgkin's lymphoma; DLBCL, Diffuse large B cell lymphoma; HL, Hodgkin's lymphoma; SLE, systemic lupus erythematosus.

### Example 2

### Introduction

The discovery and characterization of lineage specific markers has been instrumental for the identification of cell subsets that underlie the complexity of the immune system. Cell surface markers, such as CDR (pan T cell marker), CD4 (helper T cells), CD8 (cytotoxic T cells), and B220/CD45R (B cells), are routinely used to differentiate lymphocyte populations [1-2]. Advances in flow cytometry labeling techniques led to the characterization of CD4 subtypes (Th1, Th2, Th17 and Treg cells) based on the detection of lineage-specific transcription factors [3]. The discovery of regulatory ‘B10 cells’ was based on the identification of a small subset of B cells that are CD1d^{hi}CD5⁺ and secrete IL-10 [4-6]. In addition, lineage specific surface markers (such as the B cell marker CD20), represent useful targets for the development of therapeutic mAbs that have proven effective against various lymphomas as well as autoimmune diseases like Rheumatoid Arthritis (RA¹) through their ability to delete pathogenic B cells [7-8].

### TSPAN33 is a Novel B Cell Activation Marker

We sought to identify novel markers of human leukocytes. To this end, we analyzed a comprehensive database of human gene expression from 105 different human tissues including cells of the immune system (known as the Body Index of Gene Expression (BIGE) database) [9-10]. This database is useful for the identification of novel genes associated with specific organs or cells [11]. We identified a gene (Tspan33) that encodes a transmembrane protein not previously associated with B cells. The tetraspanin superfamily is defined by a conserved domain structure (Pfam00335) with a cysteine-rich long extracellular loop (LEL) containing a highly conserved cysteine-cysteine-glycine (CCG) motif [12]. These features facilitate the formation of large molecular complexes with other proteins, such as integrins or other tetraspanins and mediate diverse functions including proliferation, adhesion, motility, and differentiation. Some tetraspanins are widely expressed in adult tissues while others, (including CD82, CD151 and CD37), exhibit a more limited expression profile and are highly expressed in specific cell lineages of the immune system [13].

Previous reports on TSPAN33

TSPAN33 has been previously reported as Penumbra (proerythroblast nu membrane), since it was originally detected in a subpopulation of erythrocyte progenitors in murine bone marrow suggesting that it was involved in hematopoiesis [14]. Tspan33 expression in the mouse bone marrow was detected in the TER 119+ fraction of bone marrow cells (erythroblasts), but not in neutrophils, T cells, monocytes, NK cells, or (resting) B cells [14]. Indeed, it is expressed in mouse pre-CFU erythroid cells and in mouse bone marrow [15]. These results may be explained by the small contribution that these Tspan33+ erythrocyte progenitors make to total bone marrow RNA. Interestingly, Heikens et al. [14] generated a Tspan33−/− mouse, and some of these mice displayed abnormal erythropoiesis within 3 months and splenomegaly at 1 year of age. However, as we show here, the expression of TSPAN33 in normal human bone marrow is very low (FIG. 3) and is instead specifically and strongly expressed by activated B lymphocytes.

### Approach

We have confirmed the expression of TSPAN33 in both mouse and human B cells. Taken together, these results indicate that TSPAN33 is a novel marker of activated B cells. In contrast to other B cell specific antigens (i.e. CD20, CD19) that are present on both resting and activated B cells, TSPAN33 is only expressed by activated B cells. We next sought to determine if TSPAN33 was also expressed in human diseases that involved activated malignant B cells. To this end we measured TSPAN33 expression in Hodgkin's lymphoma (HL), various types of non-Hodgkin's lymphoma (NHL), and in two autoimmune diseases, systemic lupus erythematosus (SLE) and rheumatoid arthritis (RA).

### Example 3

### Methods

### Microarray Analyses

The generation of the Body Index of Gene Expression database (BIGE) has been described [9-10]. Briefly, total RNAs were obtained from 4 male and 4 female human donors, between 3-5 hours post-mortem or augmented with commercially available human tissue RNAs (Clontech, Palo Alto, Calif.). Genome-wide gene expression data was obtained using Affymetrix Human Genome U133 Plus 2.0 gene arrays (Affymetrix, Santa Clara, Calif.) and data normalization, and summarization were done in ArrayAssist software (Iobion Labs, La Jolla, Calif.).

qRT-PCR

RNA was isolated from human cell lines/cells or tissue using the QiagenRNeasy® kit according to the manufacturer's instructions (Qiagen, CA). The RNA was converted to cDNA using the QuantiTect® Reverse Transcription (Qiagen, CA). qPCR was performed using the Roche LightCycler® 480 Real-Time PCR system with probes designed to detect TSPAN33, CD19, CD20, CD138 and GAPDH (Roche, Pleasanton, Calif.). Primers for TSPAN33 having the sequences in SEQ ID NOs: 4 to 5 were used.

### Detection of TSPAN33 Protein

Polyclonal rabbit antibodies against human beta actin (Santa Cruz biotech, Santa Cruz, Calif.), beta tubulin (MP Biomedicals, Santa Ana, Calif.) and Tspan33/TSPAN33 (Abcam, Cambridge, Mass.) were used for western blotting.

### Cell Lines

The human B cell line 2E2 has been described [16]. The human T cell line Jurkat, was obtained from the ATCC (American Type Culture Collection, Manassas, Va.). The murine cell line A20-2J has been described [17]. All DLBCL lines were a kind gift of David Fruman (UC Irvine Institute for Immunology). PBMCs from human donors were isolated by Ficoll density gradient. Mouse spleen B cells were enriched using Ficoll density gradient separation followed by panning with anti-CD3 mAb (Biolegend, San Diego, Calif.) and anti-CD11c mAb (Biolegend) coated plates. Briefly, 10 cm tissue culture plates were coated with anti-CD3 and anti-CD11c for 2 hours at 37° C. Splenocytes isolated by Ficoll density gradient separation were incubated on the coated plates for 2 hours and the non-adherent cells were collected and passed through a second round of enrichment.

### Reagents

B cells were stimulated using either LPS (Sigma Aldrich, St Louis, Mo.)+mouse or human rIL-4 (Sigma), anti-CD40 mAb clone G38.5 (Invitrogen, Carlsbad, Calif.)+rIL-4 or CpG+pokeweed mitogen (PWM)+pansorbin (Sigma). T cells were stimulated using anti-CD3 mAb+anti-CD28 mAb (Biolegend) or phorbol 12-myristate 13-acetate (PMA)+ionomycin (Sigma).

### Mice

C57B1/6j (stock number 000664) and MRL/fas^{lpr/lpr} mice (stock number 000485) were obtained from the Jackson Laboratory (Bar Harbor, Me.). All animal protocols were approved by the Institutional Animal Care and Use Committee (IACUC) of the University of California, Irvine.

### Human Samples

Human PBMC's were obtained from peripheral blood by venipucture from Lupus patients or normal subjects. This protocol was approved by the Institutional Review Board (IRB) of the INNCMSZ and the samples were obtained following informed consent. Lupus patients fulfilled at least four 1982 American Rheumatism Association revised criteria for SLE [18]. Clinical disease activity was scored using the SLE Disease Activity Index or SLEDAI [19]. Controls had inactive disease (SLEDAI<3) and patients with active disease with indices above 3 were considered as having active disease. cDNA was prepared using the M-MLV reverse transcriptase according to the manufacturer's instructions (Invitrogen, Carlsbad, Calif.).

### Tissue Array

Human tissue samples for immunohistochemistry were obtained from autopsies and represent archival samples from the Anatomy and Pathology Service of the University Hospital of the UANL. Tissue arrays were performed on normal human kidney or human lymphoma biopsies, including 6 HL patients, 6 Follicular lymphoma patients, 6 DLBCL patients, and 2 mantle cell lymphoma, following antigen retrieval (demasking) using protease and/or heat treatment as described [20]. Sections were then stained using anti-TSPAN33 antibodies followed by secondary donkey anti-rabbit IgG enzyme conjugates (Abcam).

### Statistical Analyse

The statistical significance was calculated using the student's T-test. Values of p<0.05 were considered statistically significant. Error bars indicate standard deviation (SD).

### Example 4

### TSPAN33 is Highly Expressed in Activated B Cells

We identified TSPAN33 as a B cell activation-specific marker through the analysis of its expression in the BIGE database (FIG. 3). Its expression profile indicates specific and restricted expression, with the highest levels observed in peripheral blood B cells activated with anti-CD40 and IL-4, followed by kidney (Table I lists the top ten sites of Tspan33 expression; the complete list is shown in supplementary information (SI 1)). The Tspan33 expression pattern from the BIGE database was confirmed using qRT-PCR on human RNAs (SI 2A) with low or undetectable expression in most other tissues including bone marrow, thymus and spleen.

TABLE 1
Top ten sites of TSPAN33 expression in humans.

| Sample | Average intensity |
|---|---|
| B cells, activated | 985.4 |
| Kidney | 526 |
| Kidney medulla | 519.1 |
| Kidney cortex | 471.3 |
| B cells, resting | 305.1 |
| Salivary gland | 244.1 |
| Monocytes, activated (LPS + IFNγ) | 238.5 |
| Tonsil | 218 |
| Pituitary gland | 189.3 |

Table shows the top ten sites of TSPAN33 expression ranking from highest to lowest average intensity. The data is derived from the BIGE database shown in FIG. 3

To confirm the microarray data, we performed qRT-PCR for Tspan33 mRNA on human B cells isolated from PBMCs, under resting or activating conditions (anti-CD40+IL-4) as well as human bone marrow (FIG. 4A). Although Tspan33 was initially identified as expressed in a subset of erythrocyte progenitors in mouse bone marrow [14], we did not detect significant Tspan33 expression in human bone marrow (FIG. 4A). Tspan33 levels in activated B cells are over 40-fold higher than either resting B cells (p=0.0204) or whole bone marrow. Since Tspan33 has only recently been studied, there are not many reagents available (including antibodies). However, we obtained an anti-TSPAN33 polyclonal antibody (Abcam) that worked in Western blot and immunohistochemistry (IHC) (following epitope retrieval) but not for FACS analyses (data not shown). Using this antibody, we observed a significant increase in TSPAN33 protein expression in activated human PBMCs (FIG. 4B). Densitometric analyses revealed a ˜5 fold increase in TSPAN33 protein expression in stimulated versus unstimulated PBMC samples.

The human 2E2 B cell line is a model for inducible B cell activation and differentiation [16]. It expresses IgM and IgD in a non-stimulated state and it readily upregulates activation-induced cytidine deaminase (Aicda) to induce class switching to downstream isotypes (a measure of activation) [21-22] following stimulation with anti-CD40 mAb+IL-4. Using qRT-PCR we observed a significant increase in Tspan33 mRNA levels following stimulation with anti-CD40+IL-4 for 12 hours compared with unstimulated 2E2 cells (p=0.013) (FIG. 4C), and the elevated Tspan33 transcript levels remained high for up to 120 hours after stimulation. Conversely, Tspan33 expression was not detectable in resting, anti-CD3+ anti-CD28 or PMA+ionomycin-stimulated Jurkat cells (human T cell leukemia). The increased expression of Tspan33 in 2E2 cells was confirmed by western blot, with a >3 fold increase (by densitometry) observed when using a polyclonal anti-Tspan33 antibody (FIG. 4D). Tspan33 expression was also measured in mouse tissue using qRT-PCR (SI 2B) and the results confirmed the human expression profile. We also observed a dose-dependent increase in Tspan33 mRNA expression in the murine B cell line A20-2J upon stimulation with increasing concentrations of LPS+IL-4 and measured by qRT-PCR (FIG. 4E). Tspan33 transcription increased in A20-2J over 50 fold (p=0.0014) with 0.1 ng/mL LPS+IL-4 stimulation and over 100 fold with 1 ng/mL or 10 ng/ml LPS+IL-4 (p=0.011 and p=0.045). Additionally, mouse spleen B cells were isolated by Ficoll density gradient separation and enriched by panning with anti-CD3 and anti-CD11c [23]. The enriched B cells were stimulated with 10 ng/mL of LPS+IL-4 for 12 hours and analyzed for Tspan33 expression by qRT-PCR. As shown in FIG. 4F, there was a ˜4 fold increase in Tspan33 transcription following LPS stimulation compared to resting conditions (p=0.00003). We should note that we also performed qRT-PCR on total mouse splenocytes under various stimulation conditions and significant upregulation of Tspan33 expression was observed when splenocytes were stimulated with CD40L+IL-4 or with anti-IgD+IL-4, but not with anti-CD3+anti-CD28 (which stimulates T cells)(data not shown). Taken together, these results indicate that TSPAN33 is a novel marker of activated B cells in both mouse and human.

### TSPAN33 is Expressed by Malignant B Cells

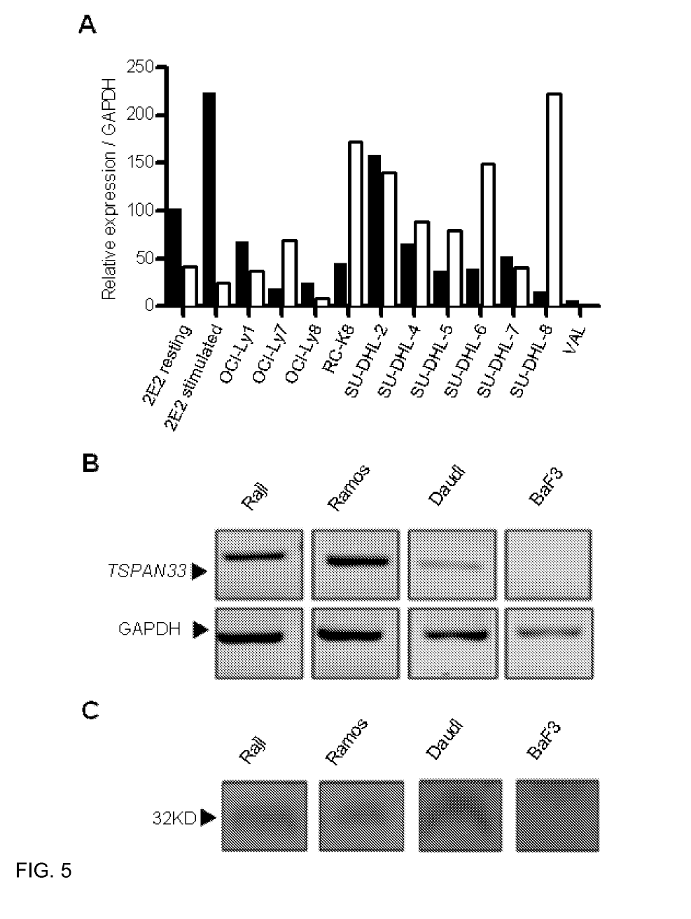

B cell activation markers are important as diagnostic tools, since elevated levels of some of these molecules, such as serum levels of sCD23, sCD27, sCD30, sCD44, CXCL13, IL-6 and IL-10 [24-25] have been reported to be associated with cancer (for example, NHL). Other known B cell antigens (i.e. CD19 and CD20) are also highly expressed in NHL [26]. We therefore hypothesized that TSPAN33 would also be expressed in human lymphomas. To test this, we performed qRT-PCR for Tspan33 expression and compared it to ms4a1 (CD20) in 11 lines including NHL cell lines characterized as DLBCL (OCI-LY1, OCI-LY7, OCI-LY8, RC-K8, SU-DHL-2, SU-DHL4, SU-DHL-5, SU-DHL-6, SU-DHL-7, and SU-DHL-8 and VAL), along with non-stimulated or stimulated (anti-CD40 mAb+IL-4) 2E2 cells (FIG. 5A). DLBCL is the most common type of aggressive NHL and represents a heterogeneous group of lymphomas with a common characteristic of diffuse proliferation of large B cells with nuclei at least twice the size of normal lymphocytes [27]. DLBCL may include centroblast, immunoblast, or anaplastic variants (similar to highly activated Reed Sternberg cells of HL) and have a proliferative index of >90% [28]. ms4a1/CD20 mRNA levels were also measured to compare its expression with Tspan33 in these lymphoma cell lines. Both ms4a1/CD20 and Tspan33 were detected in all DLBCL lines. In fact, Tspan33 expression levels were comparable to CD20 in DLBCL.

In contrast to DLBCL, Burkitt's lymphoma (BL) has a germinal center phenotype [21], including a CD10⁺, BCL6⁺ and BCL2⁺ distinct phenotype with round, medium-sized morphology, with a proliferative index of 100% [29] and may express CD20 [28]. To explore the expression of TSPAN33 in Burkitt's lymphoma, we performed RT-PCR and western blotting on several Burkitt's lymphoma lines including Raji, Ramos, and Daudi, as well as in mouse Baf3 cells (Pro-B cell line) as a control (FIGS. 5B and 5C). TSPAN33 expression was detected at both the mRNA and protein levels in all Burkitt's lymphoma lines, but not in BaF3 cells. We conclude that TSPAN33 is also expressed in human Burkitt's lymphoma.

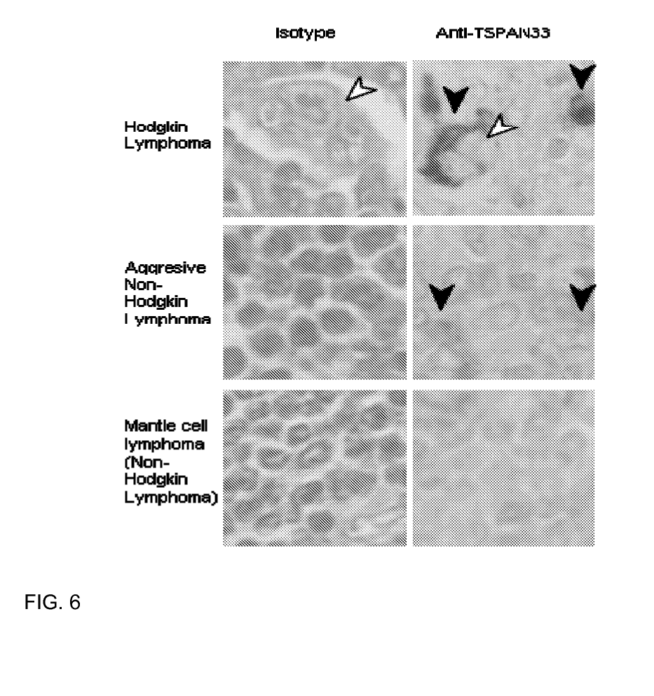

To further characterize TSPAN33 expression in other B cell lymphomas, we sought to perform immunohistochemistry (IHC) on tissue arrays prepared from biopsies of patients diagnosed with DLBCL (n=6), mantle cell lymphoma (another type of NHL, n=2), Follicular lymphoma (second most common type of indolent NHL, n=6) and HL (n=6). Table 2 and FIG. 6 show representative images of lymph nodes from patients with HL, DLBCL, or mantle cell lymphomas. Tspan33 was highly expressed in Reed-Sternberg cells (a cell characteristic of Hodgkin's Lymphoma) in HL, while DLBCL also stained positive for TSPAN33 uniformly, consistent with the qPCR data shown in FIG. 5. Mantle cell lymphoma was negative for TSPAN33 staining Reed-Sternberg cells are thought to be derived from germinal center B cells that have undergone somatic hypermutation and failed to undergo apoptosis, and therefore may represent an activated form of lymphoma [30]. DLBCL has been described above. Mantle cell lymphoma, on the other hand, is a type of mature CD5⁺ B cell lymphoma believed to originate from naïve, pre-germinal center lymphocytes, and may represent a form of non-activated B lymphocyte [31]. These differences in TSPAN33 levels may reflect the activation or differentiation state of each B cell lymphoma. On the other hand, the expression of TSPAN33 in each lymphoma suggests that it may represent another biomarker that could reflect the aggressiveness of each lymphoma or could be used as a prognostic factor [32-33].

TABLE 2
TSPAN33 expression in human lymphomas.

| | TSPAN33 | Pattern of |
|---|---|---|
| Case | positive samples | staining |
| HL | 6/6 | Localized to Reed |
| | | Sternberg cells |
| DLBCL | 6/6 | uniform |
| Mantle cell lymphoma | 0/2 | negative |

Table shows the results from the IHC staining of TSPAN33 expression on tissue arrays taken human biopsies from individual patients diagnosed with HL (n = 6), DLBCL (n = 6), and mantle cell lymphoma (n = 2). The total number of patients and staining pattern are also indicated.

### TSPAN33 is Expressed in Systemic Lupus Erythematosus and Rheumatoid Arthritis Lesions

Markers of B cell activation are also associated with certain autoimmune diseases. For example, CD25, HLA-DR, CD38, and BLyS are all elevated and associated with autoantibody production in clinical SLE [34-35]. Serum immunoglobulin levels and the B cell-associated cytokines IL-6, IL-21 and BLyS are all significantly elevated in patients with newly diagnosed RA [36-38]. Blocking BLyS reduces disease symptoms in MRL/fas^{lpr/lpr} mice (soluble TACI) [39] and also provides therapeutic benefit in humans (anti-BLyS mAb:Benlysta) [40]. To address the role of Tspan33 in autoimmune diseases, we measured Tspan33 mRNA expression in PBMCs from SLE patients, in RA synovial lesions or in a mouse model of SLE.

MRL/fas^{lpr/lpr} mice develop a spontaneous and progressive systemic autoimmune syndrome sharing many features with human SLE and RA, including dysregulated B cell activation, elevated antibody and autoantibody production, inflammation, and immune complex deposition in the kidney, which results in fatal glomerulonephritis [39-40]. The abnormal activation of B cells in MRLIfas^{lpr/lpr} mice and human SLE leads to elevated Aicda expression, resulting in pathogenic class-switched and hypermutated antibodies, which mediate tissue and organ damage [39, 41]. MRLIfas^{lpr/lpr} mice develop high titers of autoantibodies and severe kidney damage by 16 weeks of age [42]. Thus, B cells play important roles in lupus pathogenesis, through both antibody-dependent and antibody-independent mechanisms [43].

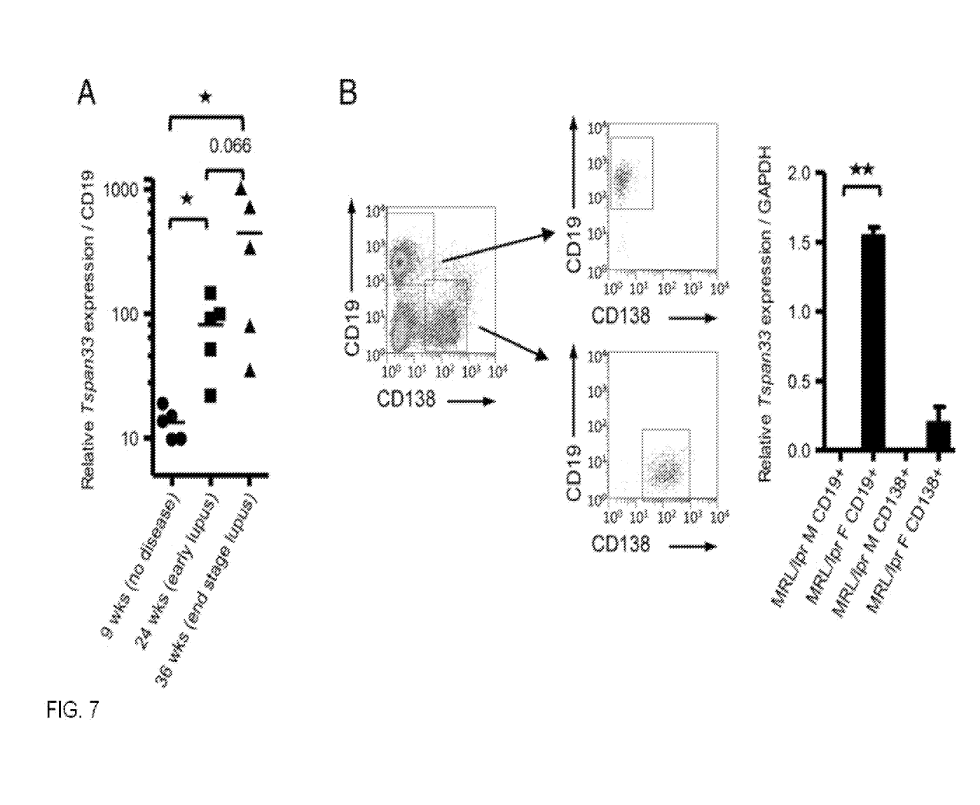

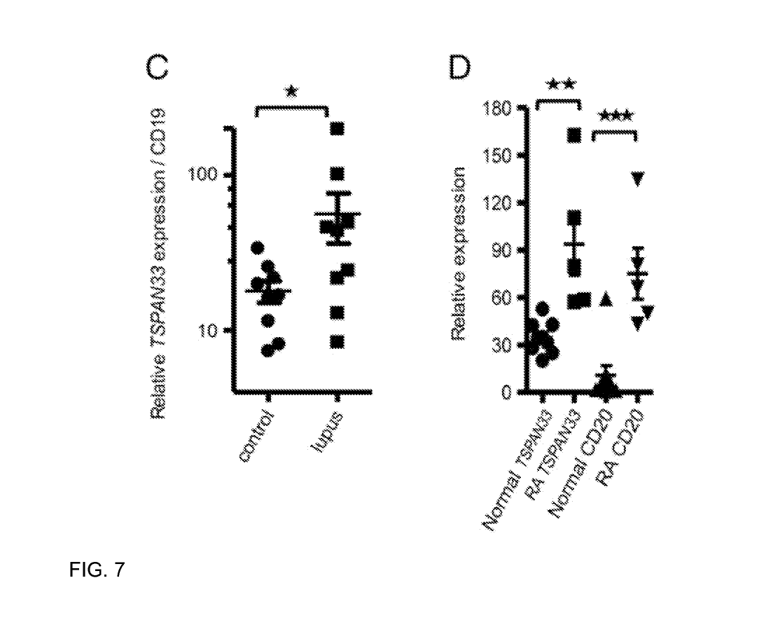

We measured Tspan33 mRNA expression in splenocytes from MRLIfas^{lpr/lpr} mice at 9, 24 and 36 weeks of age and normalized it to CD19 in order to explore the B cell contribution (FIG. 7A). We found that 24-week-old MRLIfas^{lpr/lpr} mice, which already exhibit extensive Lupus symptoms including skin lesions, autoantibodies, and renal pathology, had a ˜10-fold increase in Tspan33 mRNA expression when compared to their 9-week-old counterparts (p=0.016), which did not yet show overt signs of pathology (although some B cells may already be activated at 9 weeks, MRLIfas^{lpr/lpr} display 90% mortality by 30 weeks of age, with the few surviving mice displaying particularly dysregulated levels of cytokines and chemokines) [42]. Tspan33 transcript expression in 36-week-old MRLIfas^{lpr/lpr} mice increased further (compared to 24-week-old mice), although this increase was not statistically significant (p=0.062). Taken together, these observations strongly suggest an important role for TSPAN33 in the pathogenesis of SLE.

As B cells are not exclusively responsible for Lupus pathogenesis, we sought to determine whether TSPAN33 upregulation during Lupus disease in MRLIfas^{lpr/lpr} mice was associated with plasma cells. To address this, we FACS-sorted splenocytes from 12 week old male and female MRLIfas^{lpr/lpr} mice for CD19⁺ 138⁻ B cells and CD19⁻ CD138⁺ plasma cells and analyzed Tspan33 expression by qRT-PCR (FIG. 7B). Tspan33 expression was significantly upregulated in CD19⁺ B cells from 12-week-old female MRLIfas^{lpr/lpr} mice (p=0.004) over their male counterparts (similar to the human disease, females are more prone to lupus-like disease with an earlier onset than males in MRLIfas^{lpr/lpr} mice). Furthermore, Tspan33 was not expressed in CD138⁺ cells, indicating that its expression is restricted to activated B cells but does not extend to terminally differentiated B cells (plasma cells). Further support for this conclusion comes from the expression of plasma cell specific markers in the BIGE database. For example, B cell maturation antigen (BCMA) is a receptor for BLyS and APRIL expressed by plasma cells [44]. In the BIGE database, BCMA is strongly expressed in human tonsil, bronchus and trachea, indicating that these tissues contain significant numbers of plasma cells (data not shown); in contrast, TSPAN33 expression is low or absent in these tissues (FIG. 11 and SI 1). We conclude that TSPAN33 is unlikely to be expressed by plasma cells. This is consistent with other markers of B cell activation that decrease upon differentiation into plasma/memory cells [45-47].

To confirm a possible role of TSPAN33 activation in human SLE, we measured the expression of Tspan33 mRNA by qRT-PCR in PBMCs from 9 healthy subjects or 9 SLE patients (FIG. 7C). PBMCs from SLE patients had a >3 fold increase in Tspan33 mRNA expression (p=0.038). These results indicate that TSPAN33 is elevated in human SLE.

We next sought to explore a possible role of activated B cells in RA. To this end, we analyzed TSPAN33 mRNA expression in a RA microarray database produced from synovial membranes of patients with this disease [48]. Levels of both TSPAN33 (p=0.0019) and CD20 (p=0.0008) transcripts were elevated in RA patients (FIG. 7D). It has been reported that the top genes elevated in the RA synovial joint membranes include multiple markers of B cell activation, including immunoglobulin light and heavy chain genes, as well as genes that target B cells like BLyS and CXCL13 [48]. These observations are consistent with previous reports that have documented the role of activated B cells in RA lesions [49-50] as well as the fact that anti-CD20 (rituxan) is an effective treatment in RA [51-52].

### TSPAN33 Expression in the Kidney

As shown in FIG. 3 and SI 2 A, TSPAN33 mRNA is also detectable in the kidney by both microarray and qPCR. Given the important physiologic role of the kidney, we sought to determine the location of TSPAN33 expression within the kidneys. To this end, we performed immunohistochemistry to detect TSPAN33 in normal human kidney sections (FIGS. 8A-8D) including a section of renal tissue where lymphoid infiltrates are present (FIG. 8A). TSPAN33 staining was detected in the proximal convoluted tubules, distal convoluted tubules and collecting ducts (FIGS. 8B-8C) but not in infiltrating lymphocytes (a result consistent with previous experiments) or in the glomeruli. Higher magnification revealed that TSPAN33 is expressed at the apical membrane and granules of epithelial brush border cells of the proximal convoluted tubules (FIG. 8D). These results support TSPAN33 as a target for therapeutic antibody development, because these sites are normally not accessible to antibodies.

### Discussion

### Overview

We have found that a member of the tetraspanin family (TSPAN 33) is a B cell activation marker because it is strongly expressed in activated B cells, and is also expressed in several lymphomas and in autoimmune diseases where pathogenic B cells are involved (including SLE and RA).

### TSPAN33 as a Novel B Cell Activation Biomarker

A number of markers, including CD72, CD20, CD19, and CD24 are currently used to identify and track B cells [53]. Activated germinal center B cells have been reported to express a variety of genes, including GL7 [54], CD10 and BCL6 [55]. Other B cell activation markers such as MUM1/IRF4 and FOXP1, as well as CD23, CD69 and the systemic B cell activation markers CXCL13, sCD23, sCD27, sCD30, sCD44 have been used as markers in the diagnosis and risk assessment of NHL and RA [25, 32, 56]. Importantly, none of these activation markers are exclusively expressed on activated B cells, as they have also been associated with other immune cell types in the periphery. Therefore, TSPAN33 represents a B cell specific activation marker that may be useful as a diagnostic tool for diseases involving B cell activation. The likelihood of using TSPAN33 expression as a potential prognostic biomarker in both lymphoma and autoimmune diseases deserves further study [57-59].

TSPAN33 as a Target for Therapeutic mAbs Against Malignant B Cells

In addition to use of TSPAN33 as a B cell activation marker, TSPAN33 is the 33th member of the tetraspanin family (TSPAN33), and therefore a transmembrane protein. This makes TSPAN33 a suitable candidate for the production of anti-TSPAN33 mAbs for therapeutic purposes. CD20, a closely related protein now assigned to the membrane-spanning 4-domains superfamily (MS4A1), is an example of an important target for the production of therapeutic monoclonal antibodies that have proven effective for the treatment of B cell malignancies such as NHL, chronic lymphocytic leukemia (CLL) and also for certain autoimmune diseases including RA [51-52, 60]. However, since CD20 is expressed on both resting and activated B cells, anti-CD20 mAb therapy results in depletion of all B cells in the peripheral blood as well as 70% of B cells in the bone marrow [25, 36, 61]. Therefore the identification of a B cell marker restricted to activated B cells, such as TSPAN33, could represent an alternative strategy for the development of a “second generation” of mAbs for the treatment of B cell-associated pathologies [32]. Other tetraspanins (CD151) are being explored as possible therapeutic antibody targets [62]. Our data strongly suggest that anti-TSPAN33 therapeutic mAbs would have the important advantage of avoiding depletion of most resting B cells in the treated patients.

### Other Sites of TSPAN33 Expression

TSPAN33 has been previously reported as Penumbra (Pro Erythroblast nu membrane) because it was originally identified as a molecule expressed in a small erythrocyte progenitor population in the bone marrow [14]. Given this expression pattern, it was described to play a role in hematopoiesis. Tspan33^{-/-} mice have been described [14] and some of them developed abnormal erythrocytes at 3 months of age. Acquired pure red cell aplasia is a related condition in humans where patients lack erythroblasts and depending on the cause may be self limiting [63]. These observations suggest that temporary inhibition of TSPAN33 in humans may have limited or manageable side effects.

Another possible complication in the use of anti-TSPAN33 mAbs as human therapeutics is its expression in the kidney. Its expression pattern there, however, suggests that this will not represent a significant obstacle because Tspan33 is not expressed in the glomeruli (FIG. 8B) but is instead expressed by epithelial cells in the proximal and distal convoluted tubules (FIGS. 8B-8C). Access of antibodies to these sites is normally prevented by size exclusion, since only smaller molecular weight proteins (like albumin ˜67 KD or hemoglobin ˜68 KD) are permeable through the glomerular barrier [64]. TSPAN33 protein expression was observed in the apical surface and granules of the epithelial cells of the kidney and these cells are involved in secretion and absorption of small proteins, ions, and organic solutes (glucose and amino acids), suggesting that TSPAN33 may participate in vesicular trafficking and/or signaling during urine filtration [12]. Moreover, kidney epithelial cells have been reported to be refractory to biologically-based cytotoxic agents and kidney cell carcinomas are also resistant to ADCC (antibody dependent cellular cytotoxicity) [65]. Finally, a Tspan33^{-/-} mouse has been reported to be viable and fertile [14], indicating that absence of Tspan33 has limited physiological impact in kidney function.

### The Function of Tspan33 in B Cell Activation

Although the function of Tspan33 in B cells is currently unknown, the strong induction of Tspan33 expression upon B cell activation strongly suggests that it may be involved in B cell signaling/activation (i.e. CD9 and CD81), maturation/survival (i.e. CD37), or antigen presentation (i.e. CD63), since other B cell-expressed tetraspanins are known to participate in these processes [66-69].

### SUMMARY

We conclude that TSPAN33 represents a potentially important biomarker of activated and malignant B cells, as well as a potential target for the development of therapeutic mAbs for the treatment of several types of B cell lymphoma (DLBCL, BL, HL) as well as some autoimmune diseases associated with pathogenic B cells showing an activated B cell phenotype (SLE and RA).

### Example 5

The following publications referred to in the Examples are incorporated by reference herein:
[1] E. V. Rothenberg, J. E. Moore, M. A. Yui, Launching the T-cell-lineage developmental programme, Nat Rev Immunol, 8 (2008) 9-21.
[2] R. R. Hardy, K. Hayakawa, B cell development pathways, Annu Rev Immunol, 19 (2001) 595-621.
[3] J. Zhu, H. Yamane, W. E. Paul, Differentiation of effector CD4 T cell populations (*), Annu Rev Immunol, 28 (2010) 445-489.
[4] T. Suda, A. O'Garra, I. MacNeil, M. Fischer, M. W. Bond, A. Zlotnik, Identification of a novel thymocyte growth-promoting factor derived from B cell lymphomas, Cell Immunol, 129 (1990) 228-240.
[5] J. D. Bouaziz, K. Yanaba, T. F. Tedder, Regulatory B cells as inhibitors of immune responses and inflammation, Immunol Rev, 224 (2008) 201-214.
[6] S. I. Katz, D. Parker, J. L. Turk, B-cell suppression of delayed hypersensitivity reactions, Nature, 251 (1974) 550-551.
[7] R. Korhonen, E. Moilanen, Anti-CD20 antibody rituximab in the treatment of rheumatoid arthritis, Basic Clin Pharmacol Toxicol, 106 (2010) 13-21.
[8] M. D. Pescovitz, Rituximab, an anti-cd20 monoclonal antibody: history and mechanism of action, Am J Transplant, 6 (2006) 859-866.
[9] J. Lee, A. Hever, D. Willhite, A. Zlotnik, P. Hevezi, Effects of RNA degradation on gene expression analysis of human postmortem tissues, FASEB J, 19 (2005) 1356-1358.
[10] R. B. Roth, P. Hevezi, J. Lee, D. Willhite, S. M. Lechner, A. C. Foster, A. Zlotnik, Gene expression analyses reveal molecular relationships among 20 regions of the human CNS, Neurogenetics, 7 (2006) 67-80.
[11] P. A. Gerber, P. A. Hevezi, B. A. Buhren, C. Martinez, H. Schrumpf, M. Gasis, S. Grether-Beck, J. Krutmann, B. Homey, A. Zlotnik, Systematic identification and characterization of novel human skin-associated genes encoding membrane and secreted proteins, PlOS ONE, In Press (2013).
[12] H. T. Maecker, S. C. Todd, S. Levy, The tetraspanin superfamily: molecular facilitators, FASEB J, 11 (1997) 428-442.
[13] A. B. van Spriel, K. L. Puls, M. Sofi, D. Pouniotis, H. Hochrein, Z. Orinska, K. P. Knobeloch, M. Plebanski, M. D. Wright, A regulatory role for CD37 in T cell proliferation, J Immunol, 172 (2004) 2953-2961.
[14] M. J. Heikens, T. M. Cao, C. Morita, S. L. Dehart, S. Tsai, Penumbra encodes a novel tetraspanin that is highly expressed in erythroid progenitors and promotes effective erythropoiesis, Blood, 109 (2007) 3244-3252.
[15] J. Seita, D. Sahoo, D. J. Rossi, D. Bhattacharya, T. Serwold, M. A. Inlay, L. I. Ehrlich, J. W. Fathman, D. L. Dill, I. L. Weissman, Gene Expression Commons: an open platform for absolute gene expression profiling, PlOS ONE, 7 (2012) e40321.
[16] Z. Xu, Z. Fulop, G. Wu, E. J. Pone, J. Zhang, T. Mai, L. M. Thomas, A. Al-Qahtani, C. A. White, S. R. Park, P. Steinacker, Z. Li, J. Yates, 3rd, B. Herron, M. Otto, H. Zan, H. Fu, P. Casali, 14-3-3 adaptor proteins recruit AID to 5′-AGCT-3′-rich switch regions for class switch recombination, Nat Struct Mol Biol, 17 (2010) 1124-1135.
[17] K. J. Kim, C. Kanellopoulos-Langevin, R. M. Merwin, D. H. Sachs, R. Asofsky, Establishment and characterization of BALB/c lymphoma lines with B cell properties, J Immunol, 122 (1979) 549-554.
[18] E. M. Tan, A. S. Cohen, J. F. Fries, A. T. Masi, D. J. McShane, N. F. Rothfield, J. G. Schaller, N. Talal, R. J. Winchester, The 1982 revised criteria for the classification of systemic lupus erythematosus, Arthritis Rheum, 25 (1982) 1271-1277.
[19] C. Bombardier, D. D. Gladman, M. B. Urowitz, D. Caron, C. H. Chang, Derivation of the SLEDAI. A disease activity index for lupus patients. The Committee on Prognosis Studies in SLE, Arthritis Rheum, 35 (1992) 630-640.
[20] A. M. Burkhardt, K. P. Tai, J. P. Flores-Guiterrez, N. Vilches-Cisneros, K. Kamdar, O. Barbosa-Quintana, R. Valle-Rios, P. A. Hevezi, J. Zuniga, M. Selman, A. J. Ouellette, A. Zlotnik, CXCL17 is a mucosal chemokine elevated in idiopathic pulmonary fibrosis that exhibits broad antimicrobial activity, J Immunol, 188 (2012) 6399-6406.
[21] A. Schaffer, A. Cerutti, S. Shah, H. Zan, P. Casali, The evolutionarily conserved sequence upstream of the human Ig heavy chain S gamma 3 region is an inducible promoter: synergistic activation by CD40 ligand and IL-4 via cooperative NF-kappa B and STAT-6 binding sites, J Immunol, 162 (1999) 5327-5336.
[22] S. R. Park, H. Zan, Z. Pal, J. Zhang, A. Al-Qahtani, E. J. Pone, Z. Xu, T. Mai, P. Casali, HoxC4 binds to the promoter of the cytidine deaminase AID gene to induce AID expression, class-switch DNA recombination and somatic hypermutation, Nat Immunol, 10 (2009) 540-550.
[23] J. L. Maravillas-Montero, P. G. Gillespie, G. Patino-Lopez, S. Shaw, L. Santos-Argumedo, Myosin 1c participates in B cell cytoskeleton rearrangements, is recruited to the immunologic synapse, and contributes to antigen presentation, J Immunol, 187 (2011) 3053-3063.
[24] E. C. Breen, S. K. Hussain, L. Magpantay, L. P. Jacobson, R. Detels, C. S. Rabkin, R. A. Kaslow, D. Variakojis, J. H. Bream, C. R. Rinaldo, R. F. Ambinder, O. Martinez-Maza, B-cell stimulatory cytokines and markers of immune activation are elevated several years prior to the diagnosis of systemic AIDS-associated non-Hodgkin B-cell lymphoma, Cancer Epidemiol Biomarkers Prev, 20 (2011) 1303-1314.
[25] A. J. De Roos, D. K. Mirick, K. L. Edlefsen, A. Z. LaCroix, K. J. Kopecky, M. M. Madeleine, L. Magpantay, O. Martinez-Maza, Markers of B-cell activation in relation to risk of non-Hodgkin lymphoma, Cancer Res, 72 (2012) 4733-4743.
[26] K. C. Anderson, M. P. Bates, B. L. Slaughenhoupt, G. S. Pinkus, S. F. Schlossman, L. M. Nadler, Expression of human B cell-associated antigens on leukemias and lymphomas: a model of human B cell differentiation, Blood, 63 (1984) 1424-1433.
[27] S. Gurbuxani, J. Anastasi, E. Hyjek, Diffuse large B-cell lymphoma—more than a diffuse collection of large B cells: an entity in search of a meaningful classification, Arch Pathol Lab Med, 133 (2009) 1121-1134.
[28] P. McGowan, N. Nelles, J. Wimmer, D. Williams, J. Wen, M. Li, A. Ewton, C. Curry, Y. Zu, A. Sheehan, C. C. Chang, Differentiating between Burkitt lymphoma and CD10+ diffuse large B-cell lymphoma: the role of commonly used flow cytometry cell markers and the application of a multiparameter scoring system, Am J Clin Pathol, 137 (2012) 665-670.
[29] N. Nakamura, H. Nakamine, J. Tamaru, S. Nakamura, T. Yoshino, K. Ohshima, M. Abe, The distinction between Burkitt lymphoma and diffuse large B-Cell lymphoma with c-myc rearrangement, Mod Pathol, 15 (2002) 771-776.
[30] R. Kuppers, The biology of Hodgkin's lymphoma, Nat Rev Cancer, 9 (2009) 15-27.
[31] P. Perez-Galan, M. Dreyling, A. Wiestner, Mantle cell lymphoma: biology, pathogenesis, and the molecular basis of treatment in the genomic era, Blood, 117 (2011) 26-38.
[32] H. Nyman, M. Jerkeman, M. L. Karjalainen-Lindsberg, A. H. Banham, S. Leppa, Prognostic impact of activated B-cell focused classification in diffuse large B-cell lymphoma patients treated with R-CHOP, Mod Pathol, 22 (2009) 1094-1101.
[33] Y. Suzuki, T. Yoshida, G. Wang, T. Togano, S. Miyamoto, K. Miyazaki, K. Iwabuchi, M. Nakayama, R. Horie, N. Niitsu, Y. Sato, N. Nakamura, Association of CD20 levels with clinicopathological parameters and its prognostic significance for patients with DLBCL, Ann Hematol, 91 (2012) 997-1005.
[34] T. Dorner, C. Giesecke, P. E. Lipsky, Mechanisms of B cell autoimmunity in SLE, Arthritis Res Ther, 13 (2011) 243.
[35] P. E. Spronk, B. T. vd Gun, P. C. Limburg, C. G. Kallenberg, B cell activation in clinically quiescent systemic lupus erythematosus (SLE) is related to immunoglobulin levels, but not to levels of anti-dsDNA, nor to concurrent T cell activation, Clin Exp Immunol, 93 (1993) 39-44.
[36] J. E. Gottenberg, C. Miceli-Richard, B. Ducot, P. Goupille, B. Combe, X. Mariette, Markers of B-lymphocyte activation are elevated in patients with early rheumatoid arthritis and correlated with disease activity in the ESPOIR cohort, Arthritis Res Ther, 11 (2009) R114.
[37] J. E. Gottenberg, J. M. Dayer, C. Lukas, B. Ducot, G. Chiocchia, A. Cantagrel, A. Saraux, P. Roux-Lombard, X. Mariette, Serum IL-6 and IL-21 are associated with markers of B cell activation and structural progression in early rheumatoid arthritis: results from the ESPOIR cohort, Ann Rheum Dis, 71 (2012) 1243-1248.
[38] T. M. Seyler, Y. W. Park, S. Takemura, R. J. Bram, P. J. Kurtin, J. J. Goronzy, C. M. Weyand, BLyS and APRIL in rheumatoid arthritis, J Clin Invest, 115 (2005) 3083-3092.
[39] C. A. White, J. Seth Hawkins, E. J. Pone, E. S. Yu, A. Al-Qahtani, T. Mai, H. Zan, P. Casali, AID dysregulation in lupus-prone MRL/Fas(lpr/lpr) mice increases class switch DNA recombination and promotes interchromosomal c-Myc/IgH loci translocations: modulation by HoxC4, Autoimmunity, 44 (2011) 585-598.
[40] P. L. Cohen, R. A. Eisenberg, Lpr and gld: single gene models of systemic autoimmunity and lymphoproliferative disease, Annu Rev Immunol, 9 (1991) 243-269.
[41] H. Zan, J. Zhang, S. Ardeshna, Z. Xu, S. R. Park, P. Casali, Lupus-prone MRL/faslpr/lpr mice display increased AID expression and extensive DNA lesions, comprising deletions and insertions, in the immunoglobulin locus: concurrent upregulation of somatic hypermutation and class switch DNA recombination, Autoimmunity, 42 (2009) 89-103.
[42] J. Liu, G. Karypis, K. L. Hippen, A. L. Vegoe, P. Ruiz, G. S. Gilkeson, T. W. Behrens, Genomic view of systemic autoimmunity in MRLlpr mice, Genes Immun, 7 (2006) 156-168.
[43] O. T. Chan, L. G. Hannum, A. M. Haberman, M. P. Madaio, M. J. Shlomchik, A novel mouse with B cells but lacking serum antibody reveals an antibody-independent role for B cells in murine lupus, J Exp Med, 189 (1999) 1639-1648.
[44] C. M. Coquery, L. D. Erickson, Regulatory roles of the tumor necrosis factor receptor BCMA, Crit Rev Immunol, 32 (2012) 287-305.
[45] A. L. Shaffer, K. I. Lin, T. C. Kuo, X. Yu, E. M. Hurt, A. Rosenwald, J. M. Giltnane, L. Yang, H. Zhao, K. Calame, L. M. Staudt, Blimp-1 orchestrates plasma cell differentiation by extinguishing the mature B cell gene expression program, Immunity, 17 (2002) 51-62.
[46] M. Jourdan, A. Caraux, J. De Vos, G. Fiol, M. Larroque, C. Cognot, C. Bret, C. Duperray, D. Hose, B. Klein, An in vitro model of differentiation of memory B cells into plasmablasts and plasma cells including detailed phenotypic and molecular characterization, Blood, 114 (2009) 5173-5181.
[47] K. A. Fairfax, A. Kallies, S. L. Nutt, D. M. Tarlinton, Plasma cell development: from B-cell subsets to long-term survival niches, Semin Immunol, 20 (2008) 49-58.
[48] H. Soto, P. Hevezi, R. B. Roth, A. Pahuja, D. Alleva, H. M. Acosta, C. Martinez, A. Ortega, A. Lopez, R. Araiza-Casillas, A. Zlotnik, Gene array analysis comparison between rat collagen-induced arthritis and human rheumatoid arthritis, Scand J Immunol, 68 (2008) 43-57.
[49] G. J. Silverman, D. A. Carson, Roles of B cells in rheumatoid arthritis, Arthritis Res Ther, 5 Suppl 4 (2003) S1-6.
[50] L. Martinez-Gamboa, H. P. Brezinschek, G. R. Burmester, T. Dorner, Immunopathologic role of B lymphocytes in rheumatoid arthritis: rationale of B cell-directed therapy, Autoimmun Rev, 5 (2006) 437-442.
[51] S. Bluml, K. McKeever, R. Ettinger, J. Smolen, R. Herbst, B-cell targeted therapeutics in clinical development, Arthritis Res Ther, 15 Suppl 1 (2013) S4.
[52] A. Vancsa, Z. Szabo, S. Szamosi, N. Bodnar, E. Vegh, L. Gergely, G. Szucs, S. Szanto, Z. Szekanecz, Longterm Effects of Rituximab on B Cell Counts and Autoantibody Production in Rheumatoid Arthritis: Use of High-sensitivity Flow Cytometry for More Sensitive Assessment of B Cell Depletion, J Rheumatol, 40 (2013) 565-571.
[53] T. W. LeBien, T. F. Tedder, B lymphocytes: how they develop and function, Blood, 112 (2008) 1570-1580.
[54] Y. Naito, H. Takematsu, S. Koyama, S. Miyake, H. Yamamoto, R. Fujinawa, M. Sugai, Y. Okuno, G. Tsujimoto, T. Yamaji, Y. Hashimoto, S. Itohara, T. Kawasaki, A. Suzuki, Y. Kozutsumi, Germinal center marker GL7 probes activation-dependent repression of N-glycolylneuraminic acid, a sialic acid species involved in the negative modulation of B-cell activation, Mol Cell Biol, 27 (2007) 3008-3022.
[55] G. Goteri, G. Lucarini, A. Zizzi, A. Costagliola, F. Giantomassi, D. Stramazzotti, C. Rubini, P. Leoni, Comparison of germinal center markers CD10, BCL6 and human germinal center-associated lymphoma (HGAL) in follicular lymphomas, Diagn Pathol, 6 (2011) 97.
[56] M. Erlanson, E. Gronlund, E. Lofvenberg, G. Roos, J. Lindh, Expression of activation markers CD23 and CD69 in B-cell non-Hodgkin's lymphoma, Eur J Haematol, 60 (1998) 125-132.
[57] J. W. Gregersen, D. R. Jayne, B-cell depletion in the treatment of lupus nephritis, Nat Rev Nephrol, 8 (2012) 505-514.
[58] E. Choy, Understanding the dynamics: pathways involved in the pathogenesis of rheumatoid arthritis, Rheumatology (Oxford), 51 Suppl 5 (2012) v3-11.
[59] D. Cornec, V. Devauchelle-Pensec, G. J. Tobon, J. O. Pers, S. Jousse-Joulin, A. Saraux, B cells in Sjogren's syndrome: from pathophysiology to diagnosis and treatment, J Autoimmun, 39 (2012) 161-167.
[60] M. R. Smith, Rituximab (monoclonal anti-CD20 antibody): mechanisms of action and resistance, Oncogene, 22 (2003) 7359-7368.
[61] M. Rehnberg, S. Amu, A. Tarkowski, M. I. Bokarewa, M. Brisslert, Short- and long-term effects of anti-CD20 treatment on B cell ontogeny in bone marrow of patients with rheumatoid arthritis, Arthritis Res Ther, 11 (2009) R123.
[62] J. F. Haeuw, L. Goetsch, C. Bailly, N. Corvaia, Tetraspanin CD151 as a target for antibody-based cancer immunotherapy, Biochem Soc Trans, 39 (2011) 553-558.
[63] K. Sawada, M. Hirokawa, N. Fujishima, Diagnosis and management of acquired pure red cell aplasia, Hematol Oncol Clin North Am, 23 (2009) 249-259.
[64] B. Haraldsson, J. Nystrom, W. M. Deen, Properties of the glomerular barrier and mechanisms of proteinuria, Physiol Rev, 88 (2008) 451-487.
[65] J. E. Surfus, J. A. Hank, E. Oosterwijk, S. Welt, M. J. Lindstrom, M. R. Albertini, J. H. Schiller, P. M. Sondel, Anti-renal-cell carcinoma chimeric antibody G250 facilitates antibody-dependent cellular cytotoxicity with in vitro and in vivo interleukin-2-activated effectors, J Immunother Emphasis Tumor Immunol, 19 (1996) 184-191.
[66] A. B. van Spriel, Tetraspanins in the humoral immune response, Biochem Soc Trans, 39 (2011) 512-517.
[67] P. K. Mattila, C. Feest, D. Depoil, B. Treanor, B. Montaner, K. L. Otipoby, R. Carter, L. B. Justement, A. Bruckbauer, F. D. Batista, The actin and tetraspanin networks organize receptor nanoclusters to regulate B cell receptor-mediated signaling, Immunity, 38 (2013) 461-474.
[68] A. B. van Spriel, S. de Keijzer, A. van der Schaaf, K. H. Gartlan, M. Sofi, A. Light, P. C. Linssen, J. B. Boezeman, M. Zuidscherwoude, I. Reinieren-Beeren, A. Cambi, F. Mackay, D. M. Tarlinton, C. G. Figdor, M. D. Wright, The tetraspanin CD37 orchestrates the alpha(4)beta(1) integrin-Akt signaling axis and supports long-lived plasma cell survival, Sci Signal, 5 (2012) ra82.
[69] S. H. Petersen, E. Odintsova, T. A. Haigh, A. B. Rickinson, G. S. Taylor, F. Berditchevski, The role of tetraspanin CD63 in antigen presentation via MHC class II, Eur J Immunol, 41 (2011) 2556-2561.

Use of the singular forms “a,” “an,” and “the”, both in the claims and the description, include plural references unless the context clearly dictates otherwise.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of treating a lymphoma or leukemia in which TSPAN33 is upregulated, comprising administering an anti-TSPAN33 antibody to a patient in need of such treatment in an amount effective to treat the lymphoma or leukemia.

2. The method of claim 1, wherein the lymphoma is a Hodgkin lymphoma, a non-Hodgkin lymphoma, precursor T-cell leukemia/lymphoma, follicular lymphoma, dDiffuse large B cell lymphoma, mantle cell lymphoma, B-cell chronic lymphocytic leukemia/lymphoma, MALT lymphoma, Burkitt's lymphoma, Burkitt's lymphoma, peripheral T-cell lymphoma-Not-Otherwise-Specified, nodular sclerosis form of Hodgkin lymphoma, or mixed-cellularity subtype of Hodgkin lymphoma.

3. The method of claim 2, wherein the lymphoma is a Hodgkin lymphoma or a non-Hodgkin lymphoma.

4. The method of claim 1, wherein the administering results in a reduced number of TSPAN33+ B-cells in the patient.

5. The method of claim 1, wherein the anti-TSPAN33 antibody is a monoclonal antibody, neutralizing antibody, or humanized antibody, or a combination thereof.

6. A method of treating an immune disease in which TSPAN33 is upregulated, comprising administering an anti-TSPAN33 antibody to a patient in need of such treatment in an amount effective to treat the immune disease.

7. The method of claim 6, wherein the immune disease is an allergy or an autoimmune disease.

8. The method of claim 6, wherein the disease is rheumatoid arthritis, psoriasis, atopic dermatitis, sjogren's syndrome, autoimmune hepatitis, primary biliary cirrhosis, ulcerative colitis, Crohn's disease, scleroderma, hypersensitivity pneumonitis, autoimmune thyroditis, hashimoto thyroiditis, Graves' disease, ankylosing spondylitis, Celiac disease, idiopathic thrombocytopenic purpura, mixed connective tissue disease, multiple sclerosis, multiple myeloma, pemphigus vulgaris, temporal arteritis, vitiligo, or systemic lupus erythematosus.

9. The method of claim 8, wherein the disease is rheumatoid arthritis or systemic lupus erythematosus.

10. The method of claim 6, wherein the administering results in a reduced number of TSPAN33+ B-cells in the patient.

11. The method of any of claim 6, wherein the anti-TSPAN33 antibody is a monoclonal antibody, neutralizing antibody, or humanized antibody, or a combination thereof.

12. A method of purifying activated B-lymphocytes, comprising mixing an anti-TSPAN33 antibody with a lymphocyte-containing cell preparation, and separating lymphocytes bound by the antibody.

13. The method of claim 12, wherein the anti-TSPAN33 antibody is a monoclonal antibody, neutralizing antibody, or humanized antibody, or a combination thereof.

14. The method of claim 12, wherein the separating is by fluorescence-activated cell sorting.

15. A method of identifying an activated and/or diseased B-lymphocyte, comprising detecting upregulated expression of TSPAN33 in the lymphocyte.

16. The method of claim 15, wherein the detecting comprises
adding an anti-TSPAN33 antibody to a sample comprising proteins of the lymphocyte,
forming an immune complex between the antibody and TSPAN33 when TSPAN33 is present in the sample, and
detecting the immune complex.

17. The method of claim 15, wherein the detecting comprises
preparing cDNA from RNA of the lymphocyte,
amplifying the cDNA with primers specific for nucleotide sequences in the TSPAN33 gene, or hybridizing the cDNA to nucleotide sequences of the TSPAN33 gene, and
detecting amplified products of the amplification reaction or detecting hybrids between the cDNA and the TSPAN33 nucleotide sequences.

18. The method of claim 15, wherein the lymphocyte is from a patient, and the method further comprises administering an anti-TSPAN33 antibody to the patient when upregulated expression of TSPAN33 is detected.

19. A method of diagnosing a lymphoma or immune disease involving activated and/or diseased B-lymphocytes, comprising
analyzing a sample of a patient for the presence of an activated and/or diseased B-lymphocyte by detecting upregulated expression of TSPAN33 in a lymphocyte of the sample according to the method of claim 15,
wherein the patient is diagnosed with the lymphoma or immune disease when the activated and/or diseased B-lymphocyte is detected.

20. The method of claim 19, wherein the disease is Hodgkin lymphoma, a non-Hodgkin lymphoma, rheumatoid arthritis or systemic lupus erythematosus.
